SEQUENCE LISTING (59 sequences)

SEQ ID NO: 1 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 2 - protein, 9 aa, Homo sapiens
GTFSSLAIS

SEQ ID NO: 3 - protein, 9 aa, Homo sapiens
GTFSSLAIT

SEQ ID NO: 4 - protein, 9 aa, Homo sapiens
GTFSALAIS

SEQ ID NO: 5 - protein, 9 aa, Homo sapiens
GTFSSLAIF

SEQ ID NO: 6 - protein, 17 aa, Homo sapiens
GIIPIFGTANYAQKFQG

SEQ ID NO: 7 - protein, 17 aa, Homo sapiens
AIIPVFGTASYAQKFQG

SEQ ID NO: 8 - protein, 17 aa, Homo sapiens
GIIPIFGDASYAQKFQG

SEQ ID NO: 9 - protein, 17 aa, Homo sapiens
GIIPIFGDANYAQKLQG

SEQ ID NO: 10 - protein, 17 aa, Homo sapiens
GIIPLFGRANYAQKFQG

SEQ ID NO: 11 - protein, 17 aa, Homo sapiens
GIIPVFGQANYAQKFQG

SEQ ID NO: 12 - protein, 15 aa, Homo sapiens
ARGGSVSGTLVDFDI

SEQ ID NO: 13 - protein, 11 aa, Homo sapiens
RASQSISSWLA

SEQ ID NO: 14 - protein, 7 aa, Homo sapiens
KASSLES

SEQ ID NO: 15 - protein, 9 aa, Homo sapiens
QQYNIYPIT

SEQ ID NO: 16 - protein, 122 aa, Homo sapiens
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSS

SEQ ID NO: 17 - protein, 122 aa, Homo sapiens
QVQLVQSGAEVKKPGSSVKVSCKASGGTFSSLAISWVRQAPGQGLEWMGAIIPVFGTASYAQKFQGRVTITADESTSTAYMELSSLRSEDTAVYYCARGGSVSGTLVDFDIWGQGTMVTVSS

SEQ ID NO: 18 - protein, 122 aa, Homo sapiens
QVQLVQSGAEVKKPGSSVKVSCKASGGTFSSLAITWVRQAPGQGLEWMGGIIPIFGDASYAQKFQGRVTITADESTSTAYMELSSLRSEDTAVYYCARGGSVSGTLVDFDIWGQGTMVTVSS

SEQ ID NO: 19 - protein, 122 aa, Homo sapiens
QVQLVQSGAEVKKPGSSVKVSCKASGGTFSSLAISWVRQAPGQGLEWMGGIIPIFGDANYAQKLQGRVTMTTDTSTSTAYMELRSLRSDDTAVYYCARGGSVSGTLVDFDIWGQGTMVTVSS

SEQ ID NO: 20 - protein, 122 aa, Homo sapiens
QVQLVQSGAEVKKPGSSVKVSCKASGGTFSALAISWVRQAPGQGLEWMGGIIPLFGRANYAQKFQGRVTITADESTSTAYMELSSLRSEDTAVYYCARGGSVSGTLVDFDIWGQGTMVTVSS

SEQ ID NO: 21 - protein, 122 aa, Homo sapiens
QVQLVQSGAEVKKPGSSVKVSCKASGGTFSSLAIFWVRQAPGQGLEWMGGIIPVFGQANYAQKFQGRVTITVDESTSTAYMELSSLRSEDTAVYYCARGGSVSGTLVDFDIWGQGTMVTVSS

SEQ ID NO: 22 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIK

SEQ ID NO: 23 - protein, 9 aa, Homo sapiens
YTFTSYYMH

SEQ ID NO: 24 - protein, 17 aa, Homo sapiens
IINPSGGSTSYAQKFQG

SEQ ID NO: 25 - protein, 15 aa, Homo sapiens
ARGGAEYIPAEYFQH

SEQ ID NO: 26 - protein, 11 aa, Homo sapiens
RASQSVSSYLA

SEQ ID NO: 27 - protein, 7 aa, Homo sapiens
DASNRAT

SEQ ID NO: 28 - protein, 8 aa, Homo sapiens
QQRPFLPT

SEQ ID NO: 29 - protein, 122 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGAEYIPAEYFQHWGQGTLVTVSS

SEQ ID NO: 30 - protein, 106 aa, Homo sapiens
EIVMTQSPATLSLSPGERATLSCRASQSVSSYLAWYQQKPGQAPRLLIYDASNRATGIPARFSGSGSGTDFTLTISSLEPEDFAVYYCQQRPFLPTFGGGTKVEIK

SEQ ID NO: 31 - protein, 9 aa, Homo sapiens
FTFSSYGMH

SEQ ID NO: 32 - protein, 9 aa, Homo sapiens
FTFASYGMH

SEQ ID NO: 33 - protein, 9 aa, Homo sapiens
FTFPSYGMH

SEQ ID NO: 34 - protein, 17 aa, Homo sapiens [sequence omitted]

SEQ ID NO: 35 - protein, 17 aa, Homo sapiens
VIWYDASTKYYADSVKG

SEQ ID NO: 36 - protein, 17 aa, Homo sapiens
VIWYDAINKYYADSVKG

SEQ ID NO: 37 - protein, 17 aa, Homo sapiens
VIWYDAVNKYYADSVKG

SEQ ID NO: 38 - protein, 17 aa, Homo sapiens
VIWYDALNKYYADSVKG

SEQ ID NO: 39 - protein, 14 aa, Homo sapiens
ARDLGYGDYAAHDY

SEQ ID NO: 40 - protein, 11 aa, Homo sapiens
RASQSISSWLA

SEQ ID NO: 41 - protein, 7 aa, Homo sapiens
KASSLES

SEQ ID NO: 42 - protein, 9 aa, Homo sapiens
QQHNTHPYT

SEQ ID NO: 43 - protein, 121 aa, Homo sapiens
QVQLVESGGGVVQPGRSLRLSCAASGFTFSSYGMHWVRQAPGKGLEWVAVIWYDGSNKGYADSVKGRFTISRDNSKNTLYLQMNSLRAEDTAVYYCARDLGYGDYAAHDYWGQGTLVTVSS

SEQ ID NO: 44 - protein, 121 aa, Homo sapiens
EVQLVESGGGLVKPGGSLRLSCAASGFTFASYGMHWVRQAPGKGLEWVAVIWYDASTKYYADSVKGRFTISRDNSKNTLYLQMNSLRAEDTAVYYCARDLGYGDYAAHDYWGQGTLVTVSS

SEQ ID NO: 45 - protein, 121 aa, Homo sapiens
QVQLVESGGGLVQPGGSLRLSCAASGFTFPSYGMHWVRQAPGKGLEWVAVIWYDAINKYYADSVKGRFTISRDNSKNTLYLQMNSLRAEDTAVYYCARDLGYGDYAAHDYWGQGTLVTVSS

SEQ ID NO: 46 - protein, 121 aa, Homo sapiens
QVQLVESGGGVVQPGRSLRLSCAASGFTFPSYGMHWVRQAPGKGLEWVAVIWYDAVNKYYADSVKGRFTISRDNSKNTLYLQMNSLRAEDTAVYYCARDLGYGDYAAHDYWGQGTLVTVSS

SEQ ID NO: 47 - protein, 121 aa, Homo sapiens
QVQLVESGGGVVQPGRSLRLSCAASGFTFPSYGMHWVRQAPGKGLEWVAVIWYDASTKYYADSVKGRFTISRDNSKNTLYLQMNSLRAEDTAVYYCARDLGYGDYAAHDYWGQGTLVTVSS

SEQ ID NO: 48 - protein, 121 aa, Homo sapiens
EVQLLESGGGLVQPGGSLRLSCAASGFTFPSYGMHWVRQAPGKGLEWVAVIWYDALNKYYADSVKGRFTISRDNSKNTLYLQMNSLRAEDTAVYYCARDLGYGDYAAHDYWGQGTLVTVSS

SEQ ID NO: 49 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQHNTHPYTFGGGTKVEIK

SEQ ID NO: 50 - protein, 452 aa, Homo sapiens
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 214 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 14 aa, Homo sapiens
  REGION 1..14: note = amino acids 150-163 of SEQ ID NO:1
YQCVQGYRALHRGP

SEQ ID NO: 53 - protein, 21 aa, Homo sapiens
  REGION 1..21: note = amino acids 166-186 of SEQ ID NO: 1
SVCKMTHGKTRWTQPQLICTG

SEQ ID NO: 54 - protein, 15 aa, Homo sapiens
  REGION 1..15: note = amino acids 42-56 of SEQ ID NO: 1
KEGTMLNCECKRGFR

SEQ ID NO: 55 - protein, 19 aa, Homo sapiens
  REGION 1..19: note = amino acids 70-88 of SEQ ID NO: 1
NSSHSSWDNQCQCTSSATR

SEQ ID NO: 56 - protein, 9 aa, Homo sapiens
  REGION 1..9: note = amino acids 150-158 of SEQ ID NO: 1
YQCVQGYRA

SEQ ID NO: 57 - protein, 5 aa, Homo sapiens
  REGION 1..5: note = amino acids 176-180 of SEQ ID NO: 1
RWTQP

SEQ ID NO: 58 - protein, 11 aa, Homo sapiens
  REGION 1..11: note = amino acids 74-84 of SEQ ID NO: 1
SSWDNQCQCTS

SEQ ID NO: 59 - protein, 15 aa, Homo sapiens
  REGION 1..15: note = amino acids 70-84 of SEQ ID NO: 1
NSSHSSWDNQCQCTS

(19) United States
(12) Patent Application Publication
(10) Pub. No.: US 2025/0257141 A1
(43) Pub. Date: Aug. 14, 2025

(54) COMBINATION THERAPY FOR THE TREATMENT OF CANCER COMPRISING A FAS AXIS ANTAGONIST AND A T-REG CELL DEPLETING AGENT ANTAGONIST

(71) Applicants: Hoffmann-La Roche Inc., Little Falls, NJ (US); Cancer Research Technology Limited, London (GB)

(72) Inventors: Maria Amann, Duebendorf (CH); Vaios Karanikas, Zurich (CH); Gabriel Schnetzler, Basel (CH); Kane Andrew Foster, London (GB); Ehsan Ghorani, London (GB); Sergio Quezada, London (GB); Chen Qing, London (GB)

(21) Appl. No.: 18/859,717

(22) PCT Filed: Apr. 26, 2023
(86) PCT No.: PCT/EP2023/060899

§ 371 Date: Oct. 24, 2024

(30) Foreign Application Priority Data
Apr. 26, 2022 (EP) 22170123.8

(51) Int. Cl. C07K 16/28; A61K 39/00; A61K 40/42; A61P 35/00

(52) U.S. Cl. CPC C07K 16/2866; A61K 39/0011; A61K 40/4233; A61P 35/00; C07K 16/2875; A61K 2039/545

(57) ABSTRACT

The present disclosure is directed to the combination of a Fas axis antagonist, such as an anti-FasL antibody, and a Treg cell depletion therapy, for example an anti-CD25 antibody, optionally with a cancer vaccine, for use in the treatment of cancer.

### FIELD OF THE INVENTION

The present invention is directed to the combination of a Fas axis antagonist and a Treg cell depletion therapy for use in the treatment of cancer.

### BACKGROUND TO THE INVENTION

Depletion of Regulatory T cells (Tregs) with antibodies against CD25 has shown to be effective in various cancer mouse models, see for example as described in WO2017/174331, WO2018/167104, WO2019/175222 and Solomon I et al, Nature Cancer, vol 1, p 1153-1166 (2020). However, response rates have been found to be lower in some poorly immunogenic mouse models, with only a partial response in the reduction of tumor growth shown to be achieved in some cases.

Combination therapy for treating cancer consisting of an anti-CD25 Treg-depleting antibody and a genetically modified GM-CSF secreting tumor vaccine (GVAX) has been shown to enhance therapeutic efficacy in the context of tumor size and mouse survival in such mouse models. However, following a partial response to such combination therapy, in some situations tumor relapse has been found to subsequently occur.

Hence, whilst current anti-CD25 antibody-based therapies have been shown to be effective in the initial treatment of cancer, there is a need to provide further treatment regimens that can help prevent tumor relapse and further improve patient survival.

### SUMMARY OF THE INVENTION

The present invention relates to the combination of a Treg cell depletion therapy and a Fas axis binding antagonist for use in treating cancer.

The present inventors have found that the use of a Fas axis antagonist, such as an anti-FasL antibody, in combination with a Treg cell depleting therapy, such as an anti-CD25 antibody, and optionally with a cancer vaccine such as GVAX, provides a treatment regimen for cancer that reduces the likelihood of tumor relapse and improves patient survival.

Accordingly, in one aspect of the invention there is provided a combination of a Treg cell depletion therapy and a Fas axis antagonist for use in the treatment of cancer. The Treg cell depletion therapy and the Fas axis antagonist are for separate, simultaneous or sequential administration.

In one aspect of the invention there is provided the combination of an anti-CD25 antibody and an anti-FasL antibody for use in the treatment of cancer. The anti-CD25 antibody and the anti-FasL antibody are for separate, simultaneous or sequential administration.

A further aspect of the invention provides Treg cell depletion therapy (e.g. an anti-CD25 antibody) for use in the treatment of cancer, wherein the Treg cell depletion therapy is for use in combination with a Fas axis antagonist (e.g. an anti-FasL antibody).

A further aspect of the invention provides a Fas axis antagonist (e.g. an anti-FasL antibody) for use in the treatment of cancer, wherein the Fas axis antagonist is for use in combination with Treg cell depletion therapy (e.g. an anti-CD25 antibody).

A further aspect of the invention provides a cancer vaccine for use in the treatment of cancer, wherein the cancer vaccine is for use in combination with a Fas axis antagonist (e.g. an anti-FasL antibody) and Treg cell depletion therapy (e.g. an anti-CD25 antibody).

A further aspect of the invention provides a method for treating cancer in a subject comprising administering a therapeutically effective amount of each of Treg cell depletion therapy and a Fas axis antagonist to the subject.

A further aspect of the invention provides a method for treating cancer in a subject comprising administering a therapeutically effective amount of each of an anti-CD25 antibody and an anti-FasL antibody to the subject.

A further aspect of the invention provides a Fas axis antagonist (e.g. an anti-FasL antibody) for use in treating or preventing relapse of cancer in a subject, wherein the subject has undergone Treg cell depletion therapy for the treatment of cancer and wherein the Fas axis antagonist is for administration to the subject after the Treg cell depletion therapy.

A further aspect of the invention provides a Treg cell depletion therapy for use in treating or preventing relapse of cancer in a subject, wherein the Treg cell depletion therapy is for use in combination with at least one dose of a Fas axis antagonist (e.g. an anti-FasL antibody), wherein the Fas axis antagonist is for administration to the subject after the Treg cell depletion therapy.

A further aspect of the invention provides a method of treating or preventing relapse of cancer in a subject, wherein the subject has undergone Treg cell depletion therapy for the treatment of cancer wherein the method comprises administering at least one dose of a Fas axis antagonist (e.g. an anti-FasL antibody) to the subject after the Treg cell depletion therapy.

A further aspect of the invention provides a method of treating or preventing relapse of cancer in a subject comprising administering to the subject:
a) a priming dose of a Treg cell depletion therapy; and
b) a booster dose of Treg cell depletion therapy and a dose of a Fas axis antagonist; wherein the booster dose of the Treg cell depletion therapy is administered before the Fas axis antagonist.

A further aspect of the invention provides a priming dose of a Treg cell depletion therapy for use in treating or preventing relapse of cancer in a subject, wherein the subject is further administered a booster dose of the Treg cell depletion therapy and a dose of a Fas axis antagonist, wherein the booster dose of the Treg cell depletion therapy is administered before the Fas axis antagonist.

A further aspect of the invention provides the combination of a) a priming dose of a Treg cell depletion therapy; b) a booster dose of Treg cell depletion therapy and c) a dose of a Fas axis antagonist for use in treating or preventing relapse of cancer in a subject, wherein the priming dose of the Treg cell depletion therapy and the booster dose of the Treg cell depletion therapy are administered before the Fas axis antagonist.

A further aspect of the invention provides a method for treating or preventing relapse of cancer in a subject comprising administering to the subject:
a) a priming dose of an anti-CD25 antibody; and
b) a booster dose of the anti-CD25 antibody and a dose of an anti-FasL antibody, wherein the booster dose of the anti-CD25 antibody is administered before the anti-FasL antibody.

A further aspect of the invention provides a priming dose of an anti-CD25 antibody for use in treating or preventing relapse of cancer in a subject, wherein the subject is further administered a booster dose of the anti-CD25 antibody and a dose of an anti-FasL antibody, wherein the booster dose of the anti-CD25 antibody is administered before the anti-FasL antibody.

The present invention also provides embodiments of any of the above aspects wherein the subject is further administered a cancer vaccine, and/or the subject has also undergone cancer vaccine therapy.

The present invention also provides the use of the components or combination of components in any of the disclosed methods of treatment.

Other aspects and embodiments of the invention are described in more detail below.

### BRIEF DESCRIPTION OF FIGURES

FIG. 1—Study design in an in vivo mouse model. C57BL/6 mice were injected with 50,000 B16/BL6 melanoma cells to the right flank subcutaneously (s.c.) at day 0. Mice were divided into 4 groups (FIGS. 1A-D) and 200 μg anti-CD25^{NIB} antibody, 1×10⁶ irradiated (150 Gy) GVAX, isotype control and/or 200 μg anti-FasL (MFL3) antibody were given at the indicated time points.

FIG. 2—In vivo mouse model showing tumor growth after initial combinational therapy, comprising an anti-CD25 antibody and a cancer vaccine, followed by further treatment with: (A) Isotype Control (B) anti-FasL antibody, (C) weekly anti-CD25 antibody, and (D) anti-FasL antibody and anti-CD25 antibody.

FIG. 3—Animal survival in mouse cancer model over the indicated number of days after initial combinational therapy comprising an anti-CD25 antibody and a cancer vaccine, followed by treatment with Isotype Control, anti-FasL antibody, weekly anti-CD25 antibody, or combination of anti-FasL antibody and anti-CD25 antibody.

### DETAILED DESCRIPTION OF THE INVENTION

The present invention provides the combination of Treg cell depletion therapy and a Fas axis binding antagonist for use in the treatment of cancer in a subject. In particular the invention provides Treg cell depletion therapy, such as an anti-CD25 antibody as a first component, and a Fas axis binding antagonist, such as an anti-FasL antibody, as a second (and optionally subsequent) component, for use in the treatment of cancer in a subject. The invention also provides methods of treating cancer in a subject comprising administering a Treg cell depletion therapy, such as an anti-CD25 antibody, and administering a Fas axis binding antagonist, such as an anti-FasL antibody.

References to “combination” or “in combination” herein refer to separate, simultaneous or sequential administration, unless the context specifies otherwise.

The inventors have found that administering an anti-CD25 antibody in combination with a Fas axis binding antagonist helps prevent tumor relapse and improves the therapeutic effectiveness of the anti-CD25 antibody treatment. Tregs are known to contribute to an immune suppressive tumor microenvironment (TME). In humans, high tumour infiltration by Treg cells and, more importantly, a low ratio of effector T (Teff) cells to Treg cells, is associated with poor outcomes in multiple human cancers (Shang et al., 2015, Sci Rep. 5:15179). Conversely, a high Teff/Treg cell ratio is associated with favourable responses to immunotherapy in both humans and mice (Hodi et al., 2008, Proc. Natl. Acad. Sci. USA, 105, 3005-3010; Quezada et al., 2006, J Clin Invest. 116 (7): 1935-45). However, during anti-CD25 antibody treatment the inventors have found that in partially responsive tumors, there is a change in the tumor microenvironment (TME) over time. Characterisation of the TME in partially responsive tumors has found changes including loss of activated effector T cells, gain of resting T cells, gain of regulatory T cells (Tregs) and increase in M2-like suppressive myeloid cells. This change in the TME, in particular where there may be a loss of activated Teff cells and a gain of Treg cells over time, can potentially lead to relapse of the originally responsive tumors.

The inventors have found that blocking the Fas/FasL axis with an inhibitor of the FasL/Fas signalling system (i.e. with a Fas axis antagonist), for example with an anti-FasL antibody or an anti-Fas antibody, may inhibit Fas-mediated cell death of activated Teffs. Fas expression is higher in activated Teff cells compared to other T cells; and % Fas+ cells in activated Teff cells show an increasing trend in stable tumours prior to relapse, suggesting that activated Teff may be more susceptible to Fas-mediated cell death.

In particular, the inventors have found that the administration of an inhibitor of the FasL/Fas signalling system (i.e. with a Fas axis antagonist), for example an anti-FasL antibody or an anti-Fas antibody, in combination with Treg cell depletion therapy, for example by the administration of anti-CD25 antibody, optionally in further combination with a cancer vaccine, for example GVAX, can maintain an immunogenic tumor microenvironment (TME), in particular maintain a high Teff/Treg cell ratio, thereby enabling an improved therapeutic result to be achieved.

The treatment regimen of the invention produces an enhanced therapeutic effect as compared to the anti-CD25 administered alone, or in combination with cancer vaccine, or the Fas axis antagonist (such as an anti-FasL antibody) administered alone.

Accordingly, in a first aspect of the invention there is provided a combination comprising a Treg cell depletion therapy and a Fas axis antagonist for use in the treatment of cancer. The Treg cell depletion therapy and the Fas axis antagonist are for separate, simultaneous or sequential administration. In one embodiment the combination comprises a) a first component which comprises the Treg cell depletion therapy and b) a second component which comprises the Fas axis antagonist for separate, simultaneous or sequential use in the treatment of cancer.

The Treg cell depletion therapy may be administered in combination with one or more further therapeutic agents. Preferably the further therapeutic agent is a cancer vaccine. Therefore, in some embodiments the Treg cell depletion therapy is administered in combination with a cancer vaccine, wherein the cancer vaccine is for separate, simultaneous or sequential administration with the Treg cell depletion therapy.

Accordingly, in one embodiment of the invention there is provided a combination comprising a) a first component which comprises the Treg cell depletion therapy, b) a second component which comprises the Fas axis antagonist and c) a third component comprising a cancer vaccine for use in the treatment of cancer. The Treg cell depletion therapy, the Fas axis antagonist and the cancer vaccine are preferably administered separately. Preferably the administration of the Treg cell depletion therapy to the subject, is followed by administering a cancer vaccine to the subject, and then followed by administering a Fas axis antagonist to the subject.

Preferably the treatment regimen can involve administering multiple doses of the Treg cell depletion therapy, the Fas axis antagonist and/or the cancer vaccine.

In a preferred embodiment of the invention the Treg cell depletion therapy comprises administering an anti-CD25 antibody, preferably in combination with a cancer vaccine. Administration of the anti-CD25 antibody in combination with the cancer vaccine enhances the therapeutic efficacy of the Treg cell depletion treatment.

In a preferred embodiment of the invention the Fas axis antagonist is an anti-FasL antibody. Accordingly, in one embodiment of the invention there is provided a combination comprising a) a first component which comprises an anti-CD25 antibody, b) a second component which comprises an anti-FasL antibody and c) a third component comprising a cancer vaccine for simultaneous, separate or sequential use in the treatment of cancer.

Preferably, the Fas axis antagonist (e.g. an anti-FasL antibody) is administered after the administration of the anti-CD25 antibody and the cancer vaccine. By “administered after” it is meant that the Fas axis antagonist (e.g. an anti-FasL antibody) is preferably administered at a time period sufficiently after the administration of the anti-CD25 antibody (and optionally after administration of the cancer vaccine) for these components to achieve their desired effect. For example, the Fas axis antagonist (e.g. an anti-FasL antibody) is administered after there is a depletion of Treg cells in the subject achieved by the administration of the anti-CD25 antibody (and optionally administration of the cancer vaccine).

In a preferred embodiment the treatment regimen involves administering an anti-CD25 antibody to the subject, followed by administering a cancer vaccine, and then followed by administering a Fas axis antagonist (e.g. an anti-FasL antibody).

The treatment regimen can involve administering multiple doses of the anti-CD25 antibody, the cancer vaccine and/or the Fas axis antagonist (e.g. an anti-FasL antibody). In one embodiment the treatment regimen involves administering multiple doses of the anti-FasL antibody. In one embodiment the anti-FasL antibody is for administration in combination with a further dose of the anti-CD25 antibody, wherein the further dose of the anti-CD25 antibody is for administration before the anti-FasL antibody. In other words, the treatment regimen involves administering the first dose of the anti-FasL antibody after at least two doses of the anti-CD25 antibody have been administered. Multiple doses of the anti-CD25 antibody may be administered to maintain Treg depletion in the subject.

In one embodiment the second dose of the anti-CD25 antibody is administered at least 7 days after the first dose of the anti-CD25 antibody, preferably at least 10 days, more preferably 14 to 35 days after administering the first dose of the anti-CD25 antibody.

In one embodiment each dose of the anti-FasL antibody is administered within 20 days of a second dose and each subsequent dose of the anti-CD25 antibody. For example, the anti-FasL antibody is preferably administered after there is a depletion of Treg cells in the subject achieved by the administration of the anti-CD25 antibody. Preferably each dose of the anti-FasL antibody is administered at least 1 day, preferably at least 3 days, more preferably at least 7 days after the second and each subsequent dose of the anti-CD25 antibody has been administered.

The administration of a dose of the anti-FasL antibody after administration of the second dose of the anti-CD25 antibody is repeated multiple times, i.e. cyclically. Each administration of the anti-CD25 antibody in combination with the anti-FasL antibody may be referred to as a treatment cycle. In one embodiment each treatment cycle of an anti-CD25 antibody/anti-FasL antibody combination is repeated multiple times preferably with at least seven days between the start of each cycle, i.e. each treatment cycle comprising the administration of an anti-CD25 antibody and an anti-FasL antibody is carried out over a period of at least 7 days.

Preferably each treatment cycle is in the range of seven to 48 days, wherein the start of each treatment cycle (i.e. day 1 of the cycle) is calculated from the administration of the anti-CD25 antibody. The treatment cycle can comprise periods of rest, i.e. the treatment cycle can comprise periods of time wherein no antibodies, or optionally cancer vaccines, are administered. In some embodiments, each subsequent dose of the anti-CD25 antibody is administered once per week (Q1W), once every two weeks (Q2W), once every three weeks (Q3W) or once every six weeks (Q6W), wherein a dose of anti-FasL antibody is administered between each anti-CD25 antibody dose. Each subsequent dose of the anti-CD25 antibody is administered at a frequency to maintain Treg cell depletion in the subject.

For example, in one embodiment the anti-FasL antibody is administered 7 days after administration of each further dose of the anti-CD25 antibody, wherein administering the anti-FasL antibody and anti-CD25 antibody is repeated multiple times with time intervals of at least 14 days between administration of each anti-CD25 antibody dose. For example, the anti-CD25 antibody is administered on day 1 of each treatment cycle and the anti-FasL antibody is administered on day 8 of the treatment cycle, wherein each treatment cycle is at least 14 days, and wherein if a cancer vaccine is administered the vaccine may be administered on any one or more of days 2-7 of each treatment cycle.

The Fas axis antagonist (e.g. anti-FasL antibody) is preferably administered whilst the subject has depleted Treg levels as a result of the treatment with the anti-CD25 antibody (and optionally the cancer vaccine).

In embodiments where the treatment regimen comprises the administration of a cancer vaccine, preferably a first dose of the cancer vaccine is administered before administering a first dose of the Fas axis antagonist (e.g. anti-FasL antibody), and before the second dose of the anti-CD25 antibody.

The treatment regimen can also comprise administering multiple doses of the cancer vaccine. The one or more doses of the cancer vaccine is preferably administered after the first dose of the anti-CD25 antibody has been administered. In a further embodiment the cancer vaccine may be administered substantially simultaneously with the anti-CD25 antibody. The treatment regimen may comprise:
a first administration of the anti-CD25 antibody;
followed by administration of one or more doses of the cancer vaccine;
followed by administration of one or more alternating doses of the anti-CD25 antibody and Fas axis antagonist, wherein each further dose of the anti-CD25 antibody is for administration before the dose of the Fas axis antagonist.

Accordingly, in one embodiment the treatment regimen comprises, in the following order:
a) administering an anti-CD25 antibody;
b) administering a cancer vaccine, preferably wherein administering the cancer vaccine comprises administering multiple doses of the cancer vaccine, preferably 3 doses of the cancer vaccine;
c) administering an anti-CD25 antibody; and
d) administering a Fas axis antagonist (e.g. an anti-FasL antibody), wherein the treatment regimen comprises repeating steps c) and d) in a cyclical manner. Preferably each treatment cycle comprising steps c) to d) is carried out over a period of at least 7 days. For example, each dose of the anti-CD25 antibody and Fas axis antagonist administered in steps c) and d) is administered Q1W, Q2W, Q3W or Q6W. Preferably each subsequent dose of the anti-CD25 antibody is administered at a frequency to maintain Treg cell depletion in the subject. The Fas axis antagonist is preferably administered after there is a depletion of Treg cells in the subject achieved by the administration of the anti-CD25 antibody.

In some embodiments the further doses of the cancer vaccine may also be administered in combination with the second and subsequent doses of the anti-CD25 antibody. In other words, the treatment regimen may comprise:
a first administration of the anti-CD25 antibody;
followed by administration of one or more doses of the cancer vaccine;
followed by administration of one or more doses of an anti-CD25 antibody, a cancer vaccine and a Fas axis antagonist, wherein each further dose of the anti-CD25 antibody is for administration before the dose of the Fas axis antagonist, and wherein each further dose of the cancer vaccine is administered between each further dose of the anti-CD25 antibody and the Fas axis antagonist.

Accordingly, in one embodiment the treatment regimen comprises, in the following order:
a) administering an anti-CD25 antibody;
b) administering a cancer vaccine, preferably wherein administering the cancer vaccine comprises administering multiple doses of the cancer vaccine, preferably 3 doses of the cancer vaccine;
c) administering an anti-CD25 antibody;
d) administering a cancer vaccine; and
e) administering a Fas axis antagonist (e.g. an anti-FasL antibody), wherein the treatment regimen comprises repeating steps c) to e) in a cyclical manner. Preferably each treatment cycle comprising steps c) to e) is carried out over a period of least 7 days. For example, each dose of the components administered in steps c) to e) are administered Q1W, Q2W, Q3W or Q6W. Preferably each subsequent dose of the anti-CD25 antibody is administered at a frequency to maintain Treg cell depletion in the subject. The Fas axis antagonist is preferably administered after there is a depletion of Treg cells in the subject achieved by the administration of the anti-CD25 antibody.

In some embodiments of the invention the initial dose of the Treg cell depletion therapy, e.g. the anti-CD25 antibody, and optionally the initial one or more doses of the cancer vaccine can be considered as priming therapy. The second and subsequent doses of the Treg cell depletion therapy, e.g. subsequent anti-CD25 antibody doses, and the optional further doses of the cancer vaccine, can be considered booster therapy.

“Priming therapy” according to the invention comprises the administration of a Treg cell depletion therapy (e.g. an anti-CD25 antibody) and optionally a cancer vaccine administered after the Treg cell depletion therapy. In some embodiments the priming therapy can comprises administering multiple doses of the cancer vaccine after administration of the Treg cell depletion therapy.

A “booster cycle”, “booster therapy” or “booster treatment” or similar according to the invention comprises the administration of a Treg cell depletion therapy (e.g. an anti-CD25 antibody) and a Fas axis antagonist (e.g. an anti-FasL antibody), and optionally a cancer vaccine. Each booster cycle or therapy comprises administration of the Treg cell depletion therapy, and then optionally one or more doses of the cancer vaccine if used, and then administration of the Fas axis antagonist. The Fas axis antagonist is preferably administered after there is a depletion of Treg cells in the subject achieved by the administration of the Treg cell depletion therapy (and optionally the cancer vaccine). Preferably the invention comprises multiple rounds of the booster therapy, e.g. each booster cycle is repeated multiple times, i.e. is carried out two, three, four or more times.

Accordingly, in a further aspect of the invention there is provided a priming dose of Treg cell depletion therapy and optionally a priming dose of the cancer vaccine for use in the treatment of cancer in a subject, wherein the subject is further administered a Fas axis antagonist (e.g. anti-FasL antibody) and a booster dose of the Treg cell depletion therapy, wherein the booster dose of the Treg cell depletion therapy is administered before the Fas axis antagonist. In some embodiments the booster dose of the Treg cell depletion therapy can be administered in combination with further booster doses of the cancer vaccine.

By “administered before” it is meant that the Treg cell depletion therapy, for example the anti-CD25 antibody, is preferably administered at a time period sufficiently before the administration of the Fas axis antagonist for the Treg cell depletion therapy to achieve its desired effect, for example the depletion of Treg cells in the subject.

In one embodiment there is provided a priming dose of an anti-CD25 antibody and optionally a priming dose of the cancer vaccine for use in the treatment of cancer in a subject, wherein the subject is further administered a dose of an anti-FasL antibody and a booster dose of the anti-CD25 antibody, wherein the booster dose of the anti-CD25 antibody is administered before the anti-FasL antibody. In some embodiments the booster dose of the anti-CD25 antibody can be followed by the administration of a booster dose of the cancer vaccine before the administration of the anti-FasL antibody.

In one embodiment there is provided a dose of an anti-FasL antibody for use in the treatment of cancer in a subject, wherein the subject is first administered a priming dose of an anti-CD25 antibody, and optionally a priming dose of the cancer vaccine, and a booster dose of the anti-CD25 antibody.

A further embodiment of the invention provides a method for the treatment of cancer in a subject comprising administering to the subject:
a) a priming dose of an anti-CD25 antibody and a priming dose of the cancer vaccine; and
b) a dose of an anti-FasL antibody and a booster dose of the anti-CD25 antibody,
wherein the booster dose of the anti-CD25 antibody is administered before the anti-FasL antibody.

Preferably the anti-FasL antibody and a booster dose of the anti-CD25 antibody are administered cyclically after the administration of the priming dose of the anti-CD25 antibody. i.e. step b) is repeated two, three, four or more times. Preferably each treatment cycle is at least 7 to 48 days wherein the start of each cycle (i.e. day 1 of each cycle) is calculated from the administration of the booster dose of the anti-CD25 antibody. For example, each booster dose of the anti-CD25 antibody is administered Q1W, Q2W, Q3W or Q6W wherein a dose of anti-FasL antibody is administered between each dose of anti-CD25 antibody. The booster doses of the anti-CD25 antibody are administered at a frequency to maintain Treg cell depletion in the subject. The anti-FasL antibody is preferably administered after there is a depletion of Treg cells in the subject achieved by the administration of the anti-CD25 antibody.

Preferably the anti-FasL antibody is administered within 20 days after each booster dose of the anti-CD25 antibody. More preferably each dose of the anti-FasL antibody is administered at least 1 day, preferably at least 3 more preferably at least 7 days after each booster dose of the anti-CD25 antibody.

For example, in one embodiment the booster dose of the anti-CD25 antibody is administered on day 1 of each booster cycle and the anti-FasL antibody is administered on day 7 or later of each cycle, wherein each cycle is at least a 14 day cycle.

In one embodiment the initial priming therapy of the anti-CD25 antibody and cancer vaccine, may comprise multiple doses of the cancer vaccine.

Preferably the multiple doses of the cancer vaccine are administered after the priming dose of the anti-CD25 antibody and before administration of the subsequent doses of the anti-CD25 antibody. Preferably the first dose of the cancer vaccine is administered at least 1 day after administering the anti-CD25 antibody.

In one embodiment the first booster cycle begins at least 7 days after administering the priming dose of the anti-CD25 antibody, preferably at least 10 days, more preferably 14 to 35 days after administering the priming dose of the anti-CD25 antibody. Each booster cycle is preferably at least 7 days, more preferably each booster cycle is 7 to 48 days. Preferably the anti-CD25 antibody and anti-FasL antibody are each administered once every cycle. In some embodiments a booster dose of the cancer vaccine can be administered in combination with the booster dose of the anti-CD25 antibody, within each anti-CD25 antibody/anti-FasL antibody cycle. Preferably the booster dose of the cancer vaccine is administered between administration of each dose of the anti-CD25 antibody and the anti-FasL antibody in each booster cycle.

A further aspect of the invention relates to treating or preventing the relapse of cancer in a subject. One embodiment of the invention provides a method of treating or preventing relapse of cancer in a subject, wherein the subject has undergone Treg cell depletion therapy for the treatment of cancer wherein the method comprises administering at least one dose of a Fas axis antagonist, e.g. an anti-FasL antibody, to the subject after the Treg cell depletion therapy.

In one embodiment the invention provides a method of treating or preventing the relapse of cancer in a subject comprising:
a) administering a priming dose of the Treg cell depletion therapy;
b) administering a one or more priming doses of the cancer vaccine;
c) administering a booster dose of the Treg cell depletion therapy;
d) optionally administering a booster dose of the cancer vaccine; and,
e) administering a dose of the Fas axis antagonist,
   wherein the method comprises repeating steps c) to e) in a cyclical manner. Preferably each treatment cycle comprising steps c) to e) is carried out over a period of at least 7 days. For example, each dose of the components administered in steps c) to e) are administered Q1W, Q2W, Q3W or Q6W. Preferably each subsequent dose of the anti-CD25 antibody is administered at a frequency to maintain Treg cell depletion in the subject. The Fas axis antagonist is preferably administered after there is a depletion of Treg cells in the subject achieved by the administration of the anti-CD25 antibody (and optionally the cancer vaccine).

A further embodiment of the invention provides a Fas axis antagonist, e.g. an anti-FasL antibody, for use in treating or preventing relapse of cancer in a subject, wherein the subject has undergone Treg cell depletion therapy for the treatment of cancer and wherein the Fas axis antagonist is for administration to the subject after the Treg cell depletion therapy.

Another embodiment of the invention provides a Treg cell depletion therapy for use in treating or preventing relapse of cancer in a subject, wherein the Treg cell depletion therapy is for use in combination with at least one dose of a Fas axis antagonist, e.g. an anti-FasL antibody, wherein a Fas axis antagonist is for administration to the subject after the Treg cell depletion therapy.

A further embodiment of the invention provides the combination of a) a priming dose of a Treg cell depletion therapy; b) a booster dose of Treg cell depletion therapy and c) a dose of a Fas axis antagonist for use in treating or preventing relapse of cancer in a subject, wherein the priming dose of the Treg cell depletion therapy and the first booster dose of the Treg cell depletion therapy are administered before the Fas axis antagonist.

A further embodiment of the invention provides the combination of a) a priming dose of a Treg cell depletion therapy; b) a priming dose of a cancer vaccine, c) a booster dose of Treg cell depletion therapy and d) a dose of a Fas axis antagonist, for use in treating or preventing relapse of cancer in a subject, wherein the priming dose of the Treg cell depletion therapy, the priming dose of a cancer vaccine and the booster dose of the Treg cell depletion therapy are administered before the Fas axis antagonist, and wherein the priming dose of the cancer vaccine is administered between the priming dose of the Treg cell depletion therapy and a first booster dose of the Treg cell depletion therapy.

In some embodiments, treatment comprises repeated administration of the booster dose of the Treg cell depletion therapy and the Fas axis antagonist wherein each booster dose of the Treg cell depletion therapy is administered before each dose of the Fas axis antagonist. The repeated administration of each booster dose of the Treg cell depletion therapy and dose of a Fas axis antagonist, is carried out cyclically.

Preferably the combination further comprises e) a booster dose of a cancer vaccine, wherein each booster dose of the cancer vaccine is administered between each booster dose of the Treg cell depletion therapy and each dose of the Fas axis antagonist. The repeated administration of each booster dose of the Treg cell depletion therapy, each booster dose of the cancer vaccine and dose of a Fas axis antagonist, is carried out cyclically.

Preferably each treatment cycle is carried out over a period of at least seven days. Preferably each cycle is in the range of seven to 48 days, wherein the start of each cycle (i.e. day 1 of each cycle) is calculated from the administration of the booster dose of the anti-CD25 antibody. The booster doses of the Treg cell depletion therapy (e.g. an anti-CD25 antibody) are administered at a frequency to maintain Treg cell depletion in the subject. The Fas axis antagonist (e.g an anti-FasL antibody) is preferably administered after there is a depletion of Treg cells in the subject achieved by the administration of the Treg cell depletion therapy.

Treating or preventing relapse of cancer means to avoid the reoccurrence, return or reappearance of the cancer, after an initial response or partial response to a prior cancer therapy. For example, in the context of the treatment of a solid tumor, a relapse of the cancer may involve an increase in tumor volume.

The Treg cell depletion therapy preferably comprises the administration of an anti-CD25 antibody. In some embodiments the anti-CD25 antibody is administered in combination with a further therapeutic agent, e.g. a cancer vaccine. The combination of the anti-CD25 antibody and cancer vaccine may be administered as described for the first aspect of the invention.

The Fas axis antagonist, e.g. an anti-FasL antibody, is preferably administered in combination with a further dose of the anti-CD25 antibody, wherein the anti-FasL antibody is administered after the further dose of the anti-CD25 antibody. The combination of the anti-FasL antibody and anti-CD25 antibody, and optionally the cancer vaccine can be administered cyclically, as described for the first aspect of the invention.

A further aspect of the invention is a method of treating or preventing relapse of cancer in a subject comprising administering a therapeutically effective amount of a Treg cell depletion therapy and a therapeutically effective amount of a Fas axis antagonist to the subject.

A further aspect of the invention is the use of a Treg cell depletion therapy and a Fas axis antagonist in the manufacture of a medicament for the treatment of cancer.

A further aspect of the invention provides an anti-CD25 antibody for use in the treatment of cancer, wherein the anti-CD25 antibody is for use in combination with an anti-FasL antibody.

A further aspect of the invention provides the use of an anti-CD25 antibody in the manufacture of a medicament for the treatment of cancer, wherein the anti-CD25 antibody is for use in combination with an anti-FasL antibody.

A further aspect of the invention provides an anti-FasL antibody for use in the treatment of cancer, wherein the anti-FasL antibody is for use in combination with an anti-CD25 antibody.

A further aspect of the invention provides an anti-FasL antibody for the manufacture of a medicament for the treatment of cancer, wherein the anti-FasL antibody is for use in combination with an anti-CD25 antibody.

A further aspect of the invention provides a cancer vaccine for use in the treatment of cancer, wherein the cancer vaccine is for use in combination with an anti-FasL antibody and an anti-CD25 antibody.

A further aspect of the invention provides the use of a cancer vaccine in the manufacture of a medicament for the treatment of cancer, wherein the cancer vaccine is for use in combination with an anti-FasL antibody and an anti-CD25 antibody.

Although some aspects and embodiments of the inventions are further described with reference to the Fas axis antagonist being an anti-FasL antibody and the Treg cell depletion therapy comprising the administration of an anti-CD25 antibody, other Fas axis antagonists and Treg cell depletion therapies can also be used in the invention. The invention relates to the treatment of cancer. In one embodiment the cancer is a CD25-positive cancer. The cancer can be a haematological cancer or a solid cancer.

In some embodiments the cancer is selected from acute myeloid leukaemia, diffuse large cell B-Cell lymphoma, melanoma, non-small cell lung cancer, renal cancer, ovarian cancer, bladder cancer, sarcoma, colorectal cancer, or more generally any human cancer for which the B16 cell line may represent preclinical models for validating compounds as being useful for their therapeutic management.

In some embodiments the cancer involves a solid tumour (e.g. a solid tumour cancer) and the treatment is for treating cancer or preventing the relapse of a solid tumour. As used herein, “solid tumours” are an abnormal growth or mass of tissue that usually does not contain cysts or liquid areas, in particular, tumours and/or metastasis (wherever located) other than leukaemia or non-solid lymphatic cancers. Solid tumours may be benign or malignant. Different types of solid tumours are named for the type of cells that form them and/or the tissue or organ in which they are located. Examples of solid tumours are sarcomas (including cancers arising from transformed cells of mesenchymal origin in tissues such as cancellous bone, cartilage, fat, muscle, vascular, hematopoietic, or fibrous connective tissues), carcinomas (including tumours arising from epithelial cells), mesothelioma, neuroblastoma, retinoblastoma, etc.

Reference to “treatment”, “treat” or “treating” a cancer as used herein defines the achievement of at least one positive therapeutic effect, such as for example, reduced number of cancer cells, reduced tumour size, reduced rate of cancer cell infiltration into peripheral organs, or reduced rate of tumour metastasis or tumour growth.

Positive therapeutic effects in cancer can be measured in a number of ways (e.g. Weber (2009) J Nucl Med 50, 1S-10S). By way of example, with respect to tumour growth inhibition, according to National Cancer Institute (NCI) standards, a T/C % ratio of 42% is the minimum level of anti-tumour activity. A T/C<10% is considered a high anti-tumour activity level, with T/C (%)=Median tumour volume of the treated/Median tumour volume of the control×100. In some embodiments, the treatment achieved by a therapeutically effective amount is any of progression free survival (PFS), disease free survival (DFS) or overall survival (OS). PFS, also referred to as “Time to Tumour Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients.

Reference to “prevention”, “preventing” (or prophylaxis) as used herein refers to delaying or preventing the onset of the symptoms of the cancer. Prevention may be absolute (such that no disease occurs) or may be effective only in some individuals or for a limited amount of time.

The invention involves the use of an Fas axis binding antagonist. As used herein the term “Fas axis binding antagonist” is a molecule that inhibits the interaction of a Fas binding partner with FasL so as block the Fas/FasL signalling pathway (also referred to as the CD95/CD95L pathway) and inhibit Fas-mediated cell death. In particular the FAS axis binding antagonists for use in the invention block FasL/Fas induced apoptosis. Approaches to target the FasL/Fas signalling are discussed in Risso V et al (2022) Cell Death and Disease vol 13 A248.

FasL, also referred to as a Fas Ligand, CD95L or CD178, is a type II membrane-bound protein that belongs to the Tumor necrosis factor (TNF) family. FasL is a protein which binds to its receptor Fas (also known as FasR, APO-1 or CD95) to induce apoptosis. FasL is predominantly expressed on activated T cells and natural killer (NK) cells. The Fas-FasL signalling pathway is involved in modulating immune responses by inducing cellular apoptosis.

Fas axis binding antagonists include inhibitors of Fas, such as an anti-Fas antibody or an antigen binding fragment thereof, and inhibitors of FasL, such as an anti-FasL antibody or an antigen binding fragment thereof. Non-antibody inhibitors of the FAS axis binding antagonists are also contemplated, for example the inhibitor of FasL, asunercept (APG101), a glycosylated fusion protein comprising the extracellular domain of human CD95 linked to the Fc domain of human IgG1.

In one embodiment of the invention the Fas axis binding antagonist is an anti-FasL antibody. As used herein an “anti-FasL antibody” refers to an antibody or antibody binding fragment thereof that is capable of binding to FasL. The anti-FasL antibody is a “neutralizing” or “antagonizing” antibody by which it is meant that the antibody's binding to FasL results in the inhibition of biological activity induced by FasL polypeptides. The antibody can specifically bind to a binding site of FasL that binds to Fas to prevent FasL from binding to Fas. Preferably, the ability of the antibody to neutralize or antagonize FasL activity can be assessed by its ability to inhibit Fas-FasL mediated apoptosis. The anti-FasL antibody can suppress Fas ligand-induced apoptosis of Fas antigen-expressing cells. Activity of anti-FasL antibodies can be assessed by assays and methods known in the art.

Anti-FasL antibodies are known in the art. Examples of anti-FasL antibodies are disclosed for example in WO2003/079750, WO1996/029350, EP0842948 and EP0872488. The contents of which are incorporated herein by reference.

The Fas axis binding antagonist is administered to a subject who has undergone Treg cell depletion therapy.

As used herein “Treg cell depletion therapy” or “Treg depletion therapy” means a treatment regimen that results in the reduction of Tregs in the subject as compared to the level of Tregs in the subject before the therapy. Compounds that deplete Treg cells are known in the art. The depletion of Tregs can be measured by techniques known in the art for example as disclosed in WO2018/167104 and Simpson et al (2013) J Exp Med 210, 1695-710. The contents of which are incorporated herein by reference.

As used herein, “regulatory T cells” (“Treg”, “Treg cells”, or “Tregs”) refer to a lineage of CD4+ T lymphocytes specialized in controlling autoimmunity, allergy and infection. Typically, they regulate the activities of T cell populations, but they can also influence certain innate immune system cell types. Tregs are usually identified by the expression of the biomarkers CD4, CD25 and Foxp3. Naturally occurring Treg cells normally constitute about 5-10% of the peripheral CD4+ T lymphocytes. However, within a tumour microenvironment (i.e. tumour-infiltrating Treg cells), they can make up as much as 20-30% of the total CD4+ T lymphocyte population.

In a preferred embodiment of the invention, the Treg cell depletion therapy comprises administering an anti-CD25 antibody, alone or in combination with other therapeutic agents, to the subject. Preferably the other therapeutic agent is a cancer vaccine.

CD25 is the alpha chain of the IL-2 receptor, and is found on activated T cells, regulatory T cells, activated B cells, some NK T cells, some thymocytes, myeloid precursors and oligodendrocytes. CD25 associates with CD122 and CD132 to form a heterotrimeric complex that acts as the high-affinity receptor for IL-2. The consensus sequence of human CD25 is shown below and identified as SEQ ID NO: 1 (Uniprot accession number P01589; the extracellular domain of mature human CD25, corresponding to amino acids 22-240 is underlined).

```
                10 20 30 40
MDSYLLMWGL LTFIMVPGCQ AELCDDDPPE IPHATFKAMA
                50 60 70 80
YKEGTMLNCE CKRGFRRIKS GSLYMLCTGN SSHSSWDNQC
                90 100 110 120
QCTSSATRNT TKQVTPQPEE QKERKTTEMQ SPMQPVDQAS
              130 140 150 160
LPGHCREPPP WENEATERIY HFVVGQMVYY QCVQGYRALH
              170 180 190 200
RGPAESVCKM THGKTRWTQP QLICTGEMET SQFPGEEKPQ
              210 220 230 240
ASPEGRPESE TSCLVTTTDF QIQTEMAATM ETSIFTTEYQ
              250 260 270
VAVAGCVFLL ISVLLLSGLT WQRRQRKSRR TI
```

As used herein an “anti-CD25 antibody” or an “an antibody that binds CD25” refers to an antibody that is capable of binding to the CD25 subunit of the IL-2 receptor. This subunit is also known as the alpha subunit of the IL-2 receptor.

An anti-CD25 antibody is an antibody capable of specific binding to the CD25 subunit (antigen) of the IL-2 receptor.

“Specific binding”, “bind specifically”, and “specifically bind” are understood to mean that the antibody has a dissociation constant (Kd) for the antigen of interest of less than about 10⁻⁶ M, 10⁻⁷ M, 10⁻⁸ M, 10⁻⁹ M, 10⁻¹⁰ M, 10⁻¹¹ M, 10⁻¹² M or 10⁻¹³ M. In a preferred embodiment, the dissociation constant is less than 10⁻⁸ M, for instance in the range of 10⁻⁹ M, 10⁻¹⁰ M, 10⁻¹¹ M, 10⁻¹² M or 10⁻¹³ M.

An anti-CD25 antibody suitable for use in the invention are antibodies that are capable of depleting or reducing Treg cells.

As used herein, references to “depleted” or “depleting” (with respect to the depletion of regulatory T cells by an anti-CD25 antibody agent) it is meant that the number, ratio or percentage of Tregs is decreased relative to when the antibody is not administered. In particular embodiments of the invention as described herein, over about 5%, 10%, 20%, 30%, 40%, 50%, 60%, 70%, 80%, 90% or 99% of the regulatory T cells are depleted.

Anti-CD25 antibodies that can deplete Treg cells and are suitable for use in the invention include for example those described in WO2017/174331, WO2018/167104, WO2019/008386, WO2019/175215, WO2019/175216, WO2019/175217, WO2019/175220, WO2019/17522. WO2019/175223, WO2019/175224, WO2019/175226, the contents of which are incorporated herein by reference.

In a preferred embodiment of the invention, the anti-CD25 antibody binds FcγR with high affinity, preferably an activating receptor with high affinity. Preferably the antibody binds FcγRI and/or FcγRIIa and/or FcγRIIIa with high affinity. In a particular embodiment, the antibody binds to at least one activatory Fcγ receptor with a dissociation constant of less than about 10⁻⁶M, 10⁻⁷M, 10⁻⁸M, 10⁻⁹M or 10⁻¹⁰M.

In some embodiments, the antibody is an IgG1 antibody, preferably a human IgG1 antibody, which is capable of binding to at least one Fc activating receptor. For example, the antibody may bind to one or more receptor selected from FcγRI, FcγRIIa, FcγRIIc, FcγRIIIa and FcγRIIIb. In some embodiments, the antibody is capable of binding to FcγRIIIa. In some embodiments, the antibody is capable of binding to FcγRIIIa and FcγRIIa and optionally FcγRI. In some embodiments, the antibody is capable of binding to these receptors with high affinity, for example with a dissociation constant of less than about 10⁻⁷M, 10⁻⁸M, 10⁻⁹M or 10⁻¹⁰M.

In some embodiments, the antibody binds an inhibitory receptor, FcγRIIb, with low affinity. In some embodiments, the antibody binds FcγRIIb with a dissociation constant higher than about 10⁻⁷ M, higher than about 10⁻⁶ M or higher than about 10⁻⁵ M.

In some embodiments the anti-CD25 antibody may be afucosylated. The Fc region of the antibody can be modified to change the glycosylation profile using known techniques in the art. Available techniques to produce antibodies with absent or reduced fucosylation profiles, include commercially available technologies such as GlyMAXX (ProBiogen) and methods such as those disclosed in WO2011/035884.

In some embodiments the anti-CD25 antibody induces ADCC activity. The anti-CD25 antibody exhibits ADCC activity against CD25+ target cells. “Antibody-dependent cell-mediated cytotoxicity” (ADCC) refers to a cell-mediated reaction in which nonspecific cytotoxic cells that express Fc receptors (FcRs) (e.g. Natural Killer (NK) cells, neutrophils, and macrophages) recognize bound antibody on a target cell and thereby lead to lysis of the target cell. In some embodiments the anti-CD25 antibody induces ADCP activity. “Antibody-dependent cell-mediated phagocytosis” (ADCP) refers to a cell-mediated reaction in which phagocytes (such as macrophages) that express Fc receptors (FcRs) recognize bound antibody on a target cell and thereby lead to phagocytosis of the target cell.

The anti-CD25 antibody used in the invention may function through ADCC and ADCP activity. ADCC and ADCP can be measured using assays that are known and available in the art.

In some embodiments of the invention the anti-CD25 antibody does not inhibit the binding of Interleukin-2 (IL-2) to CD25. References herein to “does not inhibit the binding of Interleukin-2 to CD25” may alternatively be expressed as the anti-CD25 antibody is a non-IL-2 blocking antibody or a “non-blocking” antibody (with respect to the non-blocking of IL-2 binding to CD25 in the presence of the anti-CD25 antibody), i.e. the antibody does not block the binding of Interleukin-2 to CD25 and in particular does not inhibit Interleukin-2 signalling in CD25-expressing cells. References herein to a non-IL-2 blocking antibody may alternatively be expressed as an anti-CD25 antibody that “does not inhibit the binding of Interleukin-2 to CD25” or as an anti-CD25 antibody that “does not inhibit the signalling of IL-2”. References to “non-blocking”, “non-IL-2 blocking”, “does not block”, or “without blocking” and the like (with respect to the non-blocking of IL-2 binding to CD25 in the presence of the anti-CD25 antibody) include embodiments wherein the anti-CD25 antibody of the invention does not block the signalling of IL-2 via CD25. That is the anti-CD25 antibody inhibits less than 50% of IL-2 signalling compared to IL-2 signalling in the absence of the antibodies. In particular embodiments of the invention as described herein, the anti-CD25 antibody inhibits less than about 50%, 40%, 35%, 30%, preferably less than about 25% of IL-2 signalling compared to IL-2 signalling in the absence of the antibodies.

Some anti-CD25 antibodies may allow binding of IL-2 to CD25, but still block signalling via the CD25 receptor, such antibodies may also be referred to “non-blocking”, “non-IL-2 blocking”, “does not block”, or “without blocking” and the like (with respect to the non-blocking of IL-2 signalling via CD25 in the presence of the anti-CD25 antibody). The non-IL-2 blocking anti-CD25 antibodies allow binding of IL-2 to CD25 to facilitate at least 50% of the level of signalling via the CD25 receptor compared to the signalling in the absence of the anti-CD25 antibody. In particular embodiments of the invention as described herein, the anti-CD25 antibody inhibits less than about 50%, 40%, 35%, 30%, preferably less than about 25% of IL-2 signalling compared to IL-2 signalling in the absence of the antibody.

IL-2 signalling via CD25 may be measured by methods as discussed for example in WO2018/167104 and as known in the art. Comparison of IL-2 signalling in the presence and absence of the anti-CD25 antibody agent can occur under the same or substantially the same conditions.

In some embodiments, IL-2 signalling can be determined by measuring by the levels of phosphorylated STAT5 protein in cells, using a standard Stat-5 phosphorylation assay. For example, a Stat-5 phosphorylation assay to measure IL-2 signalling may involve culturing PMBC cells in the presence of the anti-CD25 antibody at a concentration of 10 μg/ml for 30 mins and then adding varying concentrations of IL-2 (for example 10 U/ml or vary concentrations of 0.25 U/ml, 0.74 U/ml, 2.22 U/ml, 6.66 U/ml or 20 U/ml) for 10 mins. Cells may then be permeabilized and levels of STAT5 protein can then be measured with a fluorescent labelled antibody to a phosphorylated STAT5 peptide analysed by flow cytometry. The percentage blocking of IL-2 signalling can be calculated as follows: % blocking=100×[(% Stat5+ cells No Antibody group−% Stat5+ cells 10 μg/ml Antibody group)/(% Stat5+ cells No Antibody group).

Examples of non-blocking anti-CD25 antibodies are described in WO2018/167104, WO2019/175215, WO2019/175216, WO2019/175217, WO2019/175220, WO2019/17522. WO2019/175223, WO2019/17524, WO2019/17526 the contents of which are incorporated herein by reference in their entirety.

The anti-CD25 antibody may specifically bind to an epitope within the extracellular region of human CD25. In some embodiments the antibody binds to an epitope that is distinct from the IL-2 binding site and does not block the binding of IL-2 to CD25.

As used herein, “epitope” refers to a portion of an antigen that is bound by an antibody or antigen-binding fragment. As is well known in the art, epitopes can be formed both from contiguous amino acids (linear epitope) or non-contiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents.

An epitope is conformational in that it is comprised of portions of an antigen that are not covalently contiguous in the antigen but that are near to one another in three-dimensional space when the antigen is in a relevant conformation. For example, for CD25, conformational epitopes are those comprised of amino acid residues that are not contiguous in CD25 extracellular domain; linear epitopes are those comprised of amino acid residues that are contiguous in CD25 extracellular domain. Means for determining the exact sequence and/or particularly amino acid residues of the epitope for the anti-CD25 antibody are known in the literature, including competition with peptides, from antigen sequences, binding to CD25 sequences from different species, truncated, and/or mutagenized (e.g. by alanine scanning or other site-directed mutagenesis), phage display-based screening, yeast presentation technologies, or (co-) crystallography techniques. Methods of determining spatial conformation of epitopes are also well known in the art and include, for example, x-ray crystallography and 2-D nuclear magnetic resonance. See, for example, Epitope Mapping Protocols in Methods in Molecular Biology, Vol. 66, Glenn E. Morris, Ed (1996). Therefore, in some embodiments the anti-CD25 antibody may recognise a conformational epitope.

In some embodiments the anti-CD25 antibody binds to an epitope wherein the epitope comprises one or more amino acid residues comprised in one or more of the amino acid stretches selected from amino acids 150-163 of SEQ ID NO:1 (YQCVQGYRALHRGP) (SEQ ID NO: 52), amino acids 166-186 of SEQ ID NO: 1 (SVCKMTHGKTRWTQPQLICTG) (SEQ ID NO: 53), amino acids 42-56 of SEQ ID NO: 1 (KEGTMLNCECKRGFR) (SEQ ID NO: 54) and amino acids 70-88 of SEQ ID NO: 1 (NSSHSSWDNQCQCTSSATR) (SEQ ID NO: 55). Preferably the epitope comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen or more amino acid residues comprised in one of more the amino acid stretches selected from amino acids 150-163 of SEQ ID NO: 1 (YQCVQGYRALHRGP) (SEQ ID NO: 52), amino acids 166-186 of SEQ ID NO: 1 (SVCKMTHGKTRWTQPQLICTG) (SEQ ID NO: 53), amino acids 42-56 of SEQ ID NO: 1 (KEGTMLNCECKRGFR) (SEQ ID NO: 54) and/or amino acids 70-88 of SEQ ID NO: 1 (NSSHSSWDNQCQCTSSATR) (SEQ ID NO: 55).

In some embodiments the anti-CD25 antibody binds to an epitope of human CD25 wherein the epitope comprises at least one sequence selected from amino acids 150-158 of SEQ ID NO: 1 (YQCVQGYRA) (SEQ ID NO: 56), amino acids 176-180 of SEQ ID NO: 1 (RWTQP) (SEQ ID NO: 57), amino acids 42-56 of SEQ ID NO: 1 (KEGTMLNCECKRGFR) (SEQ ID NO: 54) and amino acids 74-84 of SEQ ID NO: 1 (SSWDNQCQCTS) (SEQ ID NO: 58). Such antibodies do not inhibit the binding of IL-2 to CD25.

In one embodiment the anti-CD25 antibody binds to an epitope comprising the sequence of amino acids 70-84 of SEQ ID NO: 1 (NSSHSSWDNQCQCTS) (SEQ ID NO: 59).

Native antibodies and immunoglobulins are usually heterotetrameric glycoproteins of about 150,000 daltons, composed of two identical light (L) chains and two identical heavy (H) chains. Each heavy chain has at the amino terminus a variable domain (VH) followed by a number of constant domains. Each light chain has a variable domain at the amino terminus (VL) and a constant domain at the carboxy terminus.

The variable regions are capable of interacting with a structurally complementary antigenic target and are characterized by differences in amino acid sequence from antibodies of different antigenic specificity. The variable regions of either heavy or light chains contain the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions.

These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed as H1, H2, H3, L1, L2, L3) for the respective heavy (H) and light (L) chains. The CDR regions specified herein are defined according to Kabat (Kabat et al., 1977. J Biol Chem 252, 6609-6616).

In some embodiments the anti-CD25 antibody is selected from the group consisting of:
(a) an antibody or antigen binding fragment thereof comprising:
   a heavy chain variable region comprising a CDR-H1 comprising the amino acid sequence of any one of SEQ ID NOs: 2-5, a CDR-H2 comprising the amino acid sequence of any one of SEQ ID NOs: 6-11 and a CDR-H3 comprising the amino acid sequence of SEQ ID NO: 12, and
   a light chain variable region comprising a CDR-L1 comprising the amino acid sequence of SEQ ID NO: 13, CDR-L2 comprising the amino acid sequence of SEQ ID NO: 14, and a CDR-L3 comprising the amino acid sequence of SEQ ID NO: 15;
(b) an antibody or antigen binding fragment thereof comprising:
   a heavy chain variable region comprising a CDR-H1 comprising the amino acid sequence of SEQ ID NO: 23, a CDR-H2 comprising the amino acid sequence of SEQ ID NO: 24, and a CDR-H3 comprising the amino acid sequence of SEQ ID NO: 25, and
   a light chain variable region comprising a CDR-L1 comprising the amino acid sequence of SEQ ID NO: 26, a CDR-L2 comprising the amino acid sequence of SEQ ID NO: 27, and a CDR-L3 comprising the amino acid sequence of SEQ ID NO: 28; and
(c) an antibody or antigen binding fragment thereof comprising:
   a heavy chain variable region comprising a CDR-H1 comprising the amino acid sequence of any one of SEQ ID NOs: 31-33, a CDR-H2 comprising the amino acid sequence of any one of SEQ ID NOs: 34-38, and a CDR-H3 comprising the amino acid sequence of SEQ ID NO: 39, and
   a light chain variable region comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

In some embodiments the anti-CD25 antibody is selected from the group consisting of:
(a) an antibody or antigen binding fragment thereof comprising:
   a heavy chain variable region comprising a CDR-H1 comprising the amino acid sequence of SEQ ID NO: 2, a CDR-H2 comprising the amino acid sequence of SEQ ID NO: 6 and a CDR-H3 comprising the amino acid sequence of SEQ ID NO: 12; and
   a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15;
(b) an antibody or antigen binding fragment thereof comprising:
   a heavy chain variable region comprising a CDR-H1 comprising the amino acid sequence of SEQ ID NO: 2, a CDR-H2 comprising the amino acid sequence of SEQ ID NO: 7 and a CDR-H3 comprising the amino acid sequence of SEQ ID NO: 12; and
   a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15;
(c) an antibody or antigen binding fragment thereof comprising:
   a heavy chain variable region comprising a CDR-H1 comprising the amino acid sequence of SEQ ID NO: 3, a CDR-H2 comprising the amino acid sequence of SEQ ID NO: 8 and a CDR-H3 comprising the amino acid sequence of SEQ ID NO: 12; and
   a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15;
(d) an antibody or antigen binding fragment thereof comprising:
   a heavy chain variable region comprising a CDR-H1 comprising the amino acid sequence of SEQ ID NO: 2, a CDR-H2 comprising the amino acid sequence of SEQ ID NO: 9 and a CDR-H3 comprising the amino acid sequence of SEQ ID NO: 12; and
   a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15;
(e) an antibody or antigen binding fragment thereof comprising:
   a heavy chain variable region comprising a CDR-H1 comprising the amino acid sequence of SEQ ID NO: 4, a CDR-H2 comprising the amino acid sequence of SEQ ID NO: 10 and a CDR-H3 comprising the amino acid sequence of SEQ ID NO: 12; and
   a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15; and
(f) an antibody or antigen binding fragment thereof comprising:
   a heavy chain variable region comprising a CDR-H1 comprising the amino acid sequence of SEQ ID NO: 5, a CDR-H2 comprising the amino acid sequence of SEQ ID NO: 11 and a CDR-H3 comprising the amino acid sequence of SEQ ID NO: 12; and
   a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15.

In some embodiments the anti-CD25 antibody is selected from the group consisting of:
(a) an antibody comprising a heavy chain variable region comprising the amino acid sequence of any one of SEQ ID NO: 16-21 and a light chain variable region comprising the amino acid sequence of SEQ ID NO: 22;
(b) an antibody comprising a heavy chain variable region comprising the amino acid sequence of SEQ ID NO: 29 and a light chain variable region comprising the amino acid sequence of SEQ ID NO: 30; and
(c) an antibody comprising a heavy chain variable region comprising the amino acid sequence of any one of SEQ ID NO: 43-48 and a light chain variable region comprising the amino acid sequence of SEQ ID NO: 49.

In some embodiments the anti-CD25 antibody is selected from the group consisting of:
(a) an antibody comprising a heavy chain variable region comprising the amino acid sequence of SEQ ID NO: 16 and a light chain variable region comprising the amino acid sequence of SEQ ID NO: 22;
(b) an antibody comprising a heavy chain variable region comprising the amino acid sequence of SEQ ID NO: 17 and a light chain variable region comprising the amino acid sequence of SEQ ID NO: 22;
(c) an antibody comprising a heavy chain variable region comprising the amino acid sequence of SEQ ID NO: 18 and a light chain variable region comprising the amino acid sequence of SEQ ID NO: 22;
(d) an antibody comprising a heavy chain variable region comprising the amino acid sequence of SEQ ID NO: 19 and a light variable region comprising the amino acid sequence of SEQ ID NO: 22;
(e) an antibody comprising a heavy chain variable region comprising the amino acid sequence of SEQ ID NO: 20 and a light chain variable region comprising the amino acid sequence of SEQ ID NO: 22;
(f) an antibody comprising a heavy chain variable region comprising the amino acid sequence of SEQ ID NO: 21 and a light chain variable region comprising the amino acid sequence of SEQ ID NO: 22; and
(g) an antibody comprising a heavy chain variable region comprising a sequence having at least 80%, at least 85%, at least 90%, at least 95%, at least 96%, at least 97%, at least 98% or at least 99% sequence identity to any one of SEQ ID NO: 16-21 and a light chain variable region comprising a sequence having at least 80%, at least 85%, at least 90%, at least 95%, at least 96%, at least 97%, at least 98% or at least 99% sequence identity to SEQ ID NO: 22.

The SEQ ID NOs for the complementarity determining regions (HCDR1-3 and LCDR1-3), and heavy and light chain variable region of exemplified antibodies are provided in the below table:

TABLE 1

| Antibody | HCDR1 | HCDR2 | HCDR2 | VH | LCDR1 | LCDR2 | LCDR3 | VL |
|---|---|---|---|---|---|---|---|---|
| aCD25-a-686 | 2 | 6 | 12 | 16 | 13 | 14 | 15 | 22 |
| aCD25-a-686-m1 | 2 | 7 | 12 | 17 | 13 | 14 | 15 | 22 |
| aCD25-a-686-m2 | 3 | 8 | 12 | 18 | 13 | 14 | 15 | 22 |
| aCD25-a-686-m3 | 2 | 9 | 12 | 19 | 13 | 14 | 15 | 22 |
| aCD25-a-686-m4 | 4 | 10 | 12 | 20 | 13 | 14 | 15 | 22 |
| aCD25-a-686-m5 | 5 | 11 | 12 | 21 | 13 | 14 | 15 | 22 |
| aCD25-a-674 | 23 | 24 | 25 | 29 | 26 | 27 | 28 | 30 |
| aCD25-a-646 | 31 | 34 | 39 | 43 | 40 | 41 | 42 | 49 |
| aCD25-a-646-m1 | 32 | 35 | 39 | 44 | 40 | 41 | 42 | 49 |
| aCD25-a-646-m2 | 33 | 36 | 39 | 45 | 40 | 41 | 42 | 49 |
| aCD25-a-646-m3 | 33 | 37 | 39 | 46 | 40 | 41 | 42 | 49 |
| aCD25-a-646-m4 | 33 | 35 | 39 | 47 | 40 | 41 | 42 | 49 |
| aCD25-a-646-m5 | 33 | 38 | 39 | 48 | 40 | 41 | 42 | 49 |

Such antibodies are further described in WO2019/175216, WO2019/175217 and WO2019/1175222. The contents of which is incorporated herein by reference.

The antibody referred to herein as aCD25-a-686 may also be referred to as RG6292. In a preferred embodiment the anti-CD25 antibody is RG6292. The anti-CD25 antibody referred to as “RG6292”, is an afucosylated human IgG1 monoclonal antibody. RG6292 has a heavy chain sequence having the sequence of SEQ ID NO: 50 and a light chain sequence having the sequence of SEQ ID NO: 51.

Such antibodies are known to be “non-IL-2 blocking” antibodies and do not inhibit the binding of IL-2 to CD25.

Variants of the above defined antibodies can also be used. Variants of the antibodies include antibodies wherein the sequence for each CDR sequence comprises an amino acid sequence with:
(i) at least 85% identity thereto, and/or
(ii) one, two, or three amino acid substitutions relative to SEQ ID NOs: 2-15, 23-28, or 31-42.

Variants of the antibodies also include antibodies wherein the sequence for each of the light chain and heavy chains comprise an amino acid sequence with:
(i) at least 80% identity thereto, and/or
(ii) one, two, three, four or five amino acid substitutions relative to SEQ ID NOs: 16-22, 29-30 or 43-51.

For example, one embodiment of the invention provides an anti-CD25 antibody for use in the treatment of cancer selected from the group comprising:
a) antibody or antigen binding fragment thereof comprising:
   a heavy chain variable region comprising a CDR-H1 comprising the amino acid sequence having at least 85% sequence identity to any one of SEQ ID NOs: 2-5, a CDR-H2 comprising the amino acid sequence having at least 85% sequence identity to any one of SEQ ID NOs: 6-11 and a CDR-H3 comprising the amino acid sequence having at least 85% sequence identity to SEQ ID NO: 12, and
   a light chain variable region comprising a CDR-L1 comprising the amino acid sequence having at least 85% sequence identity to SEQ ID NO: 13, CDR-L2 comprising the amino acid sequence having at least 85% sequence identity to SEQ ID NO: 14, and a CDR-L3 comprising the amino acid sequence having at least 85% sequence identity to SEQ ID NO: 15;
b) antibody or antigen binding fragment thereof comprising:
   a heavy chain variable region comprising a CDR-H1 comprising the amino acid sequence having one, two, or three, amino acid substitutions relative to any one of SEQ ID NOs: 2-5, a CDR-H2 comprising the amino acid sequence having one, two, or three amino acid substitutions relative to any one of SEQ ID NOs: 6-11 and a CDR-H3 comprising the amino acid sequence having one, two, or three, amino acid substitutions relative to SEQ ID NO: 12, and
   a light chain variable region comprising a CDR-L1 comprising the amino acid sequence having one, two, or three, amino acid substitutions relative to SEQ ID NO: 13, CDR-L2 comprising the amino acid sequence having one, two, or three, amino acid substitutions relative to SEQ ID NO: 14, and a CDR-L3 comprising the amino acid sequence having one, two, or three, amino acid substitutions relative to SEQ ID NO: 15; and
c) antibody or antigen binding fragment thereof comprising:
   a heavy chain variable region comprising:
      i) an amino acid sequence having at least 80% sequence identity to any one of SEQ ID NOs: 16-21; or
      ii) an amino acid sequence having one, two, three, four or five amino acid substitutions compared to SEQ ID NOs: 16-21;
   and
   a light chain variable region comprising:
      i) an amino acid sequence having at least 80% sequence identity to SEQ ID NO: 22; or
      ii) an amino acid sequence having one, two, three, four or five amino acid substitutions compared to SEQ ID NO: 22.

Percent (%) identity as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptides or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)). The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100). Generally, references to % identity herein refer to % identity along the entire length of the molecule, unless the context specifies or implies otherwise.

As used herein, the term “antibody” refers to both intact immunoglobulin molecules as well as fragments thereof that include the antigen-binding site, and includes polyclonal, monoclonal, genetically engineered and otherwise modified forms of antibodies, including but not limited to chimeric antibodies, humanised antibodies, heteroconjugate and/or multispecific antibodies (e.g., bispecific antibodies, diabodies, tribodies, and tetrabodies), and antigen binding fragments of antibodies, including e.g. Fab′, F(ab′)₂, Fab, Fv, rIgG, polypeptide-Fc fusions, single chain variants (scFv fragments, VHHs, Trans-Bodies®, Affibodies®, shark single domain antibodies, single chain or Tandem diabodies (TandAb®), VHHs, Anticalins®, Nanobodies®, minibodies, BiTERs, bicyclic peptides and other alternative immunoglobulin protein scaffolds). In some embodiments, an antibody may lack a covalent modification (e.g., attachment of a glycan) that it would have if produced naturally. In some embodiments, an antibody may contain a covalent modification (e.g., attachment of a glycan, a detectable moiety, a therapeutic moiety, a catalytic moiety, or other chemical group providing improved stability or administration of the antibody, such as poly-ethylene glycol). In some embodiments, the antibody may be in the form of a masked antibody (e.g. Probodies®). A masked antibody can comprise a blocking or “mask” peptide that specifically binds to the antigen binding surface of the antibody and interferes with the antibody's antigen binding. The mask peptide is linked to the antibody by a cleavable linker (e.g. by a protease). Selective cleavage of the linker in the desired environment, i.e. in the tumour environment, allows the masking/blocking peptide to dissociate, enabling antigen binding to occur in the tumour, and thereby limiting potential toxicity issues. “Antibody” may also refer to camelid antibodies (heavy-chain only antibodies) and antibody-like molecules such as anticalins (Skerra (2008) FEBS J 275, 2677-83). In some embodiments, an antibody is polyclonal or oligoclonal, that is generated as a panel of antibodies, each associated to a single antibody sequence and binding more or less distinct epitopes within an antigen (such as different epitopes within human CD25 extracellular domain that are associated to different reference anti-human CD25 antibodies, or different epitopes of FasL). Polyclonal or oligoclonal antibodies can be provided in a single preparation for medical uses as described in the literature (Kearns J D et al., 2015. Mol Cancer Ther. 14:1625-36).

The antibodies used in the present invention may be monospecific, bispecific, or multispecific. “Multispecific antibodies” may be specific for different epitopes of one target antigen or polypeptide, or may contain antigen-binding domains specific for more than one target antigen or polypeptide. In some embodiments of the invention the antibody is monospecific. In some embodiments the antibody binds CD25 or FasL in a monovalent manner (i.e. a ratio of one antibody to one CD25 molecule or one FasL molecule respectively). In further embodiments the antibody is a monospecific bivalent antibody, i.e. the antibody binds CD25 in a ratio of one antibody to two CD25 molecules, or FasL in a ratio of one antibody to two FasL molecules.

In some embodiments of the invention the antibody is monoclonal. The antibody may additionally or alternatively be humanised or human. In a further embodiment, the antibody is human, or in any case an antibody that has a format and features allowing its use and administration in human subjects.

As used herein, “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced.

As used herein, “human antibody” refers to antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

Antibodies (Abs) and immunoglobulins (Igs) are glycoproteins having the same structural characteristics. Immunoglobulins may be from any class such as IgA, IgD, IgG, IgE or IgM. Immunoglobulins can be of any subclass such as IgG1, IgG2, IgG3, or IgG4. In a preferred embodiment of the invention the anti-CD25 antibody is from the IgG class, preferably the IgG1 subclass. In one embodiment, the anti-CD25 antibody is from the human IgG1 subclass. In a preferred embodiment of the invention the anti-FasL antibody is from the IgG class, preferably the IgG1 subclass. In one embodiment, the anti-FasL antibody is from the human IgG1 subclass.

The Treg cell depletion therapy can be administered in combination with a cancer vaccine. Cancer vaccines are known in the art. “Cancer vaccines” as used herein refer to therapeutic cancer vaccines administrated to cancer patients and designed to eradicate cancer cells through strengthening the patient's own immune responses. In particular the cancer vaccine will be capable of generating tumour antigen specific response. The cancer vaccine chosen will depend on the cancer type to be treated. Cancer vaccines include, but are not limited to, peptide-based vaccines, whole cell vaccines, dendritic cell vaccines, and DNA vaccines.

In one embodiment the cancer vaccine to be administered according to the invention is a cytokine-expressing cellular immunotherapy.

The term “cytokine-expressing cellular immunotherapy” as used herein refers to a composition comprising a population of cells that has been genetically modified to express a cytokine, e.g., GM-CSF. The cells of such a “cytokine-expressing cellular immunotherapy” comprise a cytokine-encoding DNA sequence operably linked to expression and control elements such that the cytokine is expressed by the cells. The cells are typically tumor cells which are irradiated to prevent further cell division. The cells may be autologous (patient specific) or allogeneic (non-patient specific) to the patient undergoing treatment.

In one embodiment of the invention a cytokine-expressing cellular immunotherapy comprises GM-CSF-expressing tumor cells. e.g. may be the cancer vaccine known as GVAX. GVAX comprises irradiated GM-CSF secreting tumor cells which activates and matures myeloid cells.

In the uses and methods described herein, the Fas axis antagonist, anti-FasL antibody, Treg cell depletion therapy, anti-CD25 antibody and the cancer vaccine are each administered in a therapeutically effective amount. As used herein, the term “therapeutically effective amount” means an amount (e.g., of an agent or of a pharmaceutical composition) that is sufficient, when administered to a population suffering from or susceptible to a disease and/or condition in accordance with a therapeutic dosing regimen, to treat such disease and/or condition. A therapeutically effective amount is one that reduces the incidence and/or severity of, stabilizes, and/or delays onset of, one or more symptoms of the disease, disorder, and/or condition in accordance with the treatment regimen. Those of ordinary skill in the art will appreciate that a “therapeutically effective amount” does not in fact require successful treatment be achieved in a particular subject.

Selection of an appropriate dosage of the antibodies will be within the capability of one skilled in the art. For example, 0.01, 0.1, 0.3, 0.5, 1, 2, 3, 4, 5, 6, 7, 8, 9, 10, 20, 30, 40, or 50 mg/kg. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). The dosage may also be varied for route of administration, the cycle of treatment, or consequently to dose escalation protocol that can be used to determine the maximum tolerated dose and dose limiting toxicity (if any) in connection to the administration of the antibody or cancer vaccine at increasing doses.

In some embodiments of the invention each of the components are administered multiple times over the treatment regimen. In some embodiments the dosing regimen may comprise a first dose of a component in a first dose amount, followed by the one or more additional doses in a second dose amount the same as the first dose amount. Alternatively, different doses of each component within a dosing regimen are of different amounts. In some embodiments, a dosing regimen comprises a first dose in a first dose amount, followed by the one or more additional doses in a second dose amount different from the first dose amount. In some embodiments the different antibodies may be administered in different dose amounts. Alternatively, the different antibodies are administered in substantially the same dose amounts. In some embodiments the same anti-CD25 antibody, anti-FasL antibody and cancer vaccine are used for each dose. Alternatively, different anti-CD25 antibodies and anti-FasL antibodies, for example different Fas axis antagonists, may be used, as part of the treatment regimen.

The antibodies and cancer vaccine according to any aspect of the invention as described herein may be in the form of a pharmaceutical composition which additionally comprises a pharmaceutically acceptable carrier, diluent or excipient. These compositions include, for example, liquid, semi-solid and solid dosage formulations, such as liquid solutions (e.g., injectable and infusible solutions), dispersions or suspensions, tablets, pills, or liposomes. In some embodiments, a preferred form may depend on the intended mode of administration and/or therapeutic application. Pharmaceutical compositions containing the antibody can be administered by any appropriate method known in the art, including, without limitation, oral, mucosal, by-inhalation, topical, buccal, nasal, rectal, or parenteral (e.g. intravenous, infusion, intratumoural, intranodal, subcutaneous, intraperitoneal, intramuscular, intradermal, transdermal, or other kinds of administration involving physical breaching of a tissue of a subject and administration of the pharmaceutical composition through the breach in the tissue). Such a formulation may, for example, be in a form of an injectable or infusible solution that is suitable for intradermal, intratumoural or subcutaneous administration, or for intravenous infusion. The administration may involve continuous dosing (e.g., perfusion) for at least a selected period of time.

In some embodiments, the antibodies can be prepared with carriers that protect it against rapid release and/or degradation, such as a controlled release formulation, such as implants, transdermal patches, and microencapsulated delivery systems. Biodegradable, biocompatible polymers can be used.

Those skilled in the art will appreciate, for example, that route of delivery (e.g., oral vs intravenous vs subcutaneous vs intratumoural, etc) may impact dose amount and/or required dose amount may impact route of delivery. For example, where particularly high concentrations of an agent within a particular site or location (e.g., within a tumour) are of interest, focused delivery (e.g., in this example, intratumoural delivery) may be desired and/or useful. Other factors to be considered when optimizing routes and/or dosing schedule for a given therapeutic regimen may include, for example, the particular cancer being treated (e.g., type, stage, location, etc.), the clinical condition of a subject (e.g., age, overall health, etc.), and other factors known to medical practitioners.

Each of the components of the combination for use according to the invention should be formulated for separate administration. The pharmaceutical compositions for each component typically should be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent. Each pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers are non-toxic to the subjects at the dosages and concentrations employed. Preferably, such a composition can further comprise a pharmaceutically acceptable carrier or excipient for use in the treatment of cancer that that is compatible with a given method and/or site of administration, for instance for parenteral (e.g. sub-cutaneous, intradermal, or intravenous injection), intratumoral, or peritumoral administration.

A further aspect of the invention provides a kit for treating a cancer. The kit comprises at least two active ingredients wherein the kit comprises:
a) a first composition comprising a Treg cell depletion therapy; and
b) a second composition comprising a Fas axis antagonist, and optionally instructions for using the compositions in a combination therapy for the treatment of cancer. The combination therapy can comprise administering the Treg cell depletion therapy; and the Fas axis antagonist in accordance with dosage regimens as described for the first and further aspects of the invention. In particular the dosage regimen can comprise administering the Fas axis antagonist after the Treg cell depletion therapy. The kit may further comprise a further active ingredient c) a third composition comprising a cancer vaccine.

The Treg cell depletion therapy, Fas axis antagonist, anti-CD25 antibody, anti-FasL antibody, cancer vaccine and dosage and treatment regimens for all further aspects of the invention can be as described for the first aspect of the invention.

Aspects and embodiments described herein with the term “comprising” may include other features or steps within the scope. It is also understood that aspects and embodiments described as “comprising” also describes aspect and embodiments wherein the term “comprising” is replaced by the term “consisting essentially of” or “consisting of”.

The phrase “selected from the group comprising” may be substituted with the phrase “selected from the group consisting of” and vice versa, wherever they occur herein.

A set of clauses defining the invention and its preferred aspects and embodiments is as follows:
1. A method for the treatment of cancer in a subject comprising administering a therapeutically effective amount of each of a Treg cell depletion therapy and a Fas axis antagonist to the subject.
2. A method according to clause 2 wherein the Treg depletion therapy comprises administering a therapeutically effective amount of an anti-CD25 antibody.
3. A method according to clause 1 or 2 wherein the method further comprises administering a cancer vaccine.
4. A method according to any one of clauses 1 to 3 wherein the Fas axis antagonist is an anti-FasL antibody.
5. A method according to any one of clauses 1 to 4 comprising administering a therapeutically effective amount of each of an anti-CD25 antibody, an anti-FasL antibody and a cancer vaccine to the subject.
6. The method according to clause 5 wherein the anti-CD25 antibody, the anti-FasL antibody and the cancer vaccine are administered separately.
7. The method according to clause 6 comprising administering the anti-FasL antibody after administration of the anti-CD25 antibody and the cancer vaccine.
8. The method according to any one of clauses 5 to 7 comprising administering the anti-FasL antibody after depletion of Treg cells in the subject by administration of the anti-CD25 antibody.
9. The method according to any one of clauses 5 to 8 wherein the method comprises administering the anti-CD25 antibody to the subject, followed by administering the cancer vaccine, and then followed by administering the anti-FasL antibody.
10. The method according to any one of clauses 5 to 9 comprising administering multiple doses of the anti-CD25 antibody and the anti-FasL antibody.
11. The method according to clause 10 comprising administering a first dose of the anti-FasL antibody after at least two doses of the anti-CD25 antibody have been administered.
12. The method according to any one of clauses 10 or 11 wherein each dose of the anti-FasL antibody is administered within 20 days of a dose of the anti-CD25 antibody.
13. The method according to any one of clauses 10 to 12 wherein each dose of the anti-FasL antibody is administered after a dose of the anti-CD25 antibody has been administered.
14. The method according to clause 13 wherein the administration of the anti-FasL antibody after the dose of the anti-CD25 antibody is repeated multiple times with time intervals of at least a week between the anti-CD25 antibody doses.
15. The method according to any one of clauses 5 to 14 comprising administering multiple doses of the cancer vaccine.
16. The method according to clause 15 wherein administering multiple doses of the cancer vaccine comprises administering a dose of the cancer vaccine between each dose of the anti-CD25 antibody and the anti-FasL antibody.
17. The method according to clause 16 comprising:
   a) administering the anti-CD25 antibody;
   b) administering the cancer vaccine, optionally wherein administering the cancer vaccine comprises administering multiple doses of the cancer vaccine;
   c) administering the anti-CD25 antibody;
   d) optionally administering the cancer vaccine; and
   e) administering the Fas axis antagonist,
wherein the method comprises repeating steps c) to e) in a treatment cycle over a period of at least 7 days.
18. The method according to any one of clauses 5 to 17 wherein the same anti-CD25 antibody is used for all doses of the anti-CD25 antibody.
19. The method according to any one of clauses 5 to 18 wherein the cancer is a CD25-positive cancer.
20. The method according to any one of clauses 5 to 19 wherein the cancer is a haematological cancer.
21. The method according to any one of clauses 5 to 20 wherein the cancer is a solid tumour.
22. The method according to any one of clauses 5 to 21 wherein the cancer is selected from acute myeloid leukaemia, diffuse large cell B-Cell lymphoma, melanoma, non-small cell lung cancer, renal cancer, ovarian cancer, bladder cancer, sarcoma, and colorectal cancer.
23. The method according to clause 22, wherein the cancer is acute myeloid leukaemia or diffuse large B-cell lymphoma.
24. The method according to clause 22, wherein the cancer is melanoma.
25. The method according to any one of clauses 5 to 24 wherein the anti-CD25 antibody inhibits less than 50% of the signalling of IL-2 via CD25 compared to IL-2 signalling in the absence of the antibody.
26. The method according to clause 25 wherein the anti-CD25 antibody inhibits less than 25% of the signalling of IL-2 via CD25 compared to IL-2 signalling in the absence of the antibody.
27. The method according to any one of clauses 5 to 26 wherein the anti-CD25 antibody is selected from the group consisting of:
   (a) an antibody comprising:
   a heavy chain variable region comprising a CDR-H1 comprising the amino acid sequence of any one of SEQ ID NOs: 2-5, a CDR-H2 comprising the amino acid sequence of any one of SEQ ID NOs: 6-11 and CDR-H3 comprising the amino acid sequence of SEQ ID NO: 12, and
   a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15;
   (b) an antibody comprising:
   a heavy chain variable region comprising a CDR-H1 comprising the amino acid sequence of SEQ ID NO: 23, a CDR-H2 comprising the amino acid sequence of SEQ ID NO: 24, a CDR-H3 comprising the amino acid sequence of SEQ ID NO: 25, and a light chain variable region comprising a CDR-L1 comprising the amino acid sequence of SEQ ID NO: 26, a CDR-L2 comprising the amino acid sequence of SEQ ID NO: 27, and a CDR-L3 comprising the amino acid sequence of SEQ ID NO: 28; and
   (c) an antibody comprising:
   a heavy chain variable region comprising a CDR-H1 comprising the amino acid sequence of any one of SEQ ID NOs: 31-33, a CDR-H2 comprising the amino acid sequence of any one of SEQ ID NOs: 34-38, a CDR-H3 comprising the amino acid sequence of SEQ ID NO: 39, and
   a light chain variable region comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.
28. The method according to any one of clauses 5 to 27 wherein the anti-CD25 antibody is RG6292.
29. The method according to any one of clauses 5 to 28 wherein the anti-CD25 antibody binds to an epitope comprising at least one sequence selected from amino acids 150-158 of SEQ ID NO: 1, amino acids 176-180 of SEQ ID NO: 1, amino acids 42-56 of SEQ ID NO: 1 and amino acids 74-84 of SEQ ID NO: 1.
30. The method according to clause 29 wherein the anti-CD25 antibody binds to an epitope comprising amino acids 70-84 of SEQ ID NO: 1.
31. The method according to any one of clauses 5 to 30 wherein the cancer vaccine is a cytokine-expressing cellular immunotherapy.
32. The method according to any one of clause 31 wherein cytokine-expressing cellular immunotherapy comprises irradiated GM-CSF-expressing tumor cells.
33. A Treg cell depletion therapy for use in the treatment of cancer, wherein the Treg cell depletion therapy antibody is for use in combination with a Fas axis antagonist and optionally a cancer vaccine.
34. An anti-CD25 antibody for use in the treatment of cancer, wherein the anti-CD25 antibody is for use in combination with an anti-FasL antibody and optionally a cancer vaccine.
35. A Fas axis antagonist for use in the treatment of cancer, wherein the Fas axis antagonist is for use in combination with Treg cell depletion therapy and optionally a cancer vaccine.
36. An anti-FasL antibody for use in the treatment of cancer, wherein the anti-FasL antibody is for use in combination with an anti-CD25 antibody and optionally a cancer vaccine.
37. A cancer vaccine for use in the treatment of cancer, wherein the cancer vaccine is for use in combination with a Fas axis antagonist and a Treg cell depletion therapy.
38. The cancer vaccine for use according to clause 37 wherein the Fas axis antagonist is an anti-FasL antibody and the Treg cell depletion therapy is an anti-CD25 antibody.
39. A combination of a Treg cell depletion therapy and a Fas axis antagonist for use in the treatment of cancer, wherein the Treg cell depletion therapy and the Fas axis antagonist are for separate, simultaneous or sequential administration.
40. The combination for use according to clause 39 wherein the Treg cell depletion therapy comprises an anti-CD25 antibody.
41. The combination for use according to clause 40 further comprising a cancer vaccine.
42. The combination for use according to clause 41 wherein the combination comprises a) a first component which comprises the anti-CD25 antibody, b) a second component which comprises the Fas axis antagonist and c) a third component which comprises the cancer vaccine for separate, simultaneous or sequential administration.
43. The combination for use according to clause 42 wherein the Fas axis antagonist is for administration after the anti-CD25 antibody and the cancer vaccine.
44. The combination for use according any one of clauses 42 to 43 wherein the Fas axis antagonist is for administration after depletion of Treg cells in the subject by administration of the anti-CD25 antibody.
45. The combination for use according any one of clauses 42 to 44 wherein the administration of the anti-CD25 antibody to the subject, is followed by administration of the cancer vaccine, and then followed by administration of the Fas axis antagonist.
46. The combination for use according to any one of clauses 42 to 45 wherein the treatment comprises administration of multiple doses of the anti-CD25 antibody.
47. The combination for use according to any one of clauses 42 to 46 wherein the treatment comprises administration of multiple doses of the Fas axis antagonist.
48. The combination for use according to any one of clauses 42 to 47 wherein the treatment comprises administration of multiple doses of the cancer vaccine.
49. The combination for use according to any one of clauses 42 to 48 wherein the administration comprises:
   a first administration of the anti-CD25 antibody;
   followed by administration of one or more doses of the cancer vaccine;
   followed by administration of one or more alternating doses of the anti-CD25 antibody and Fas axis antagonist, wherein the further dose of the anti-CD25 antibody is for administration before the Fas axis antagonist.
50. The combination for use according to clauses 49 wherein the administration of the combination of the dose of the Fas axis antagonist and the further dose of the anti-CD25 antibody is repeated in a treatment cycle over a period of at least 7 days.
51. The combination for use according to any one of clauses 49 or 50 wherein the administration of the one or more alternating doses of the anti-CD25 antibody and Fas axis antagonist also comprises administration of one or more doses of a cancer vaccine, wherein each further dose of the anti-CD25 antibody and the cancer vaccine is for administration before the dose of the Fas axis antagonist.
52. The combination for use according to any one of clauses 42 to 51 wherein the anti-CD25 antibody inhibits less than 50% of the signalling of IL-2 via CD25 compared to IL-2 signalling in the absence of the antibody.
53. The combination for use according to any one of clauses 42 to 52 wherein the anti-CD25 antibody is as defined in any one of clauses 25-30.
54. The combination for use according to any one of clauses 39 to 53 wherein the cancer vaccine is a cytokine-expressing cellular immunotherapy, preferably wherein the cytokine-expressing cellular immunotherapy comprises GM-CSF-expressing tumor cells.
55. The combination for use according to any one of clauses 42 to 54 wherein the Fas axis antagonist is an anti-FasL antibody.
56. A combination of an anti-CD25 antibody, an anti-FasL antibody and a cancer vaccine for use in the treatment of cancer, wherein the anti-CD25 antibody, the anti-FasL antibody and the cancer vaccine are for separate, simultaneous or sequential administration.
57. A method of treating or preventing relapse of cancer in a subject, wherein the subject has undergone Treg cell depletion therapy for the treatment of cancer wherein the method comprises administering at least one dose of a Fas axis antagonist to the subject after the Treg cell depletion therapy.
58. A method of according to clause 57 wherein the Fas axis antagonist is an anti-FasL antibody.
59. The method according to clause 57 or 58 wherein the Treg cell depletion therapy comprises the administration of an anti-CD25 antibody.
60. The method according to any one of clauses 57 to 59 wherein the method further comprises administering a cancer vaccine.
61. The method according to any one of clauses 57 to 60 comprising administering the Fas axis antagonist in combination with a further dose of an anti-CD25 antibody.
62. The method according to clause 61 wherein each dose of the Fas axis antagonist is administered after each further dose of the anti-CD25 antibody.
63. The method according to clause 62 wherein the method comprises in the following order:
   a) administering an anti-CD25 antibody;
   b) administering a cancer vaccine, optionally wherein administering the cancer vaccine comprises administering multiple doses of the cancer vaccine;
   c) administering an anti-CD25 antibody;
   d) administering a cancer vaccine; and
   e) administering a Fas axis antagonist,
wherein the method comprises repeating steps c) to e) in a treatment cycle of over a period of at least 7 days.
64. A method for the treatment of cancer in a subject comprising administering to the subject:
   a) a priming dose of a Treg cell depletion therapy; and
   b) a dose of a Fas axis antagonist and a booster dose of a Treg cell depletion therapy, wherein the booster dose of the Treg cell depletion therapy is administered before the Fas axis antagonist.
65. A method according to clause 64 wherein the Fas axis antagonist is an anti-FasL antibody.
66. The method according to anyone of clauses 64 or 65 comprising administering multiple doses of a Fas axis antagonist and the anti-CD25 antibody, wherein each dose of the Fas axis antagonist is administered after a booster dose of the Treg cell depletion therapy.
67. The method according to any one of clauses 64 to 66 comprising administering multiple doses of the Fas axis antagonist and the booster dose of the Treg cell depletion therapy, wherein there is at least 7 days between booster doses of the Treg cell depletion therapy.
68. The method according to any one of clauses 64 to 67 comprising administering a priming dose of a cancer vaccine in combination with the priming dose of the Treg cell depletion therapy.
69. The method according to clause 68 wherein the priming dose of the cancer vaccine is administered after the priming dose Treg cell depletion therapy and before the booster dose of Treg cell depletion therapy.
70. The method according to clause 69 comprising administering booster doses of the cancer vaccine, wherein each booster dose of the cancer vaccine is administered in combination with each booster dose of the Treg cell depletion therapy.
71. The method according to clause 70, wherein the method comprises:
   a) administering a priming dose of the Treg cell depletion therapy;
   b) administering a one or more priming doses of the cancer vaccine;
   c) administering a booster dose of the Treg cell depletion therapy;
   d) optionally administering a booster dose of the cancer vaccine; and,
   e) administering a dose of the Fas axis antagonist,
wherein the method comprises repeating steps c) to e) in a treatment cycle of a period of at least 7 days.
72. The method according to any one of clauses 64 to 71 wherein the Treg cell depletion therapy comprises an anti-CD25 antibody.
73. The method according to clause 72 wherein the anti-CD25 antibody is as defined in any one of clause 25 to 30.
74. The method according to any one of clauses 60-63, or 68 to 73 wherein the cancer vaccine is as defined in any one of clauses 31 to 32.
75. A Fas axis antagonist for use in treating or preventing relapse of cancer in a subject, wherein the subject has undergone Treg cell depletion therapy for the treatment of cancer and wherein the Fas axis antagonist is for administration to the subject after the Treg cell depletion therapy.
76. The Fas axis antagonist for use according to clause 75 wherein the Fas axis antagonist is an anti-FasL antibody.
77. Treg cell depletion therapy for use in treating or preventing relapse of cancer in a subject, wherein the Treg cell depletion therapy is for use in combination with at least one dose of a Fas axis antagonist, wherein the Fas axis antagonist is for administration to the subject after the Treg cell depletion therapy.
78. Treg cell depletion therapy for use according to clause 77 wherein the subject has already undergone an initial Treg cell depletion therapy for the treatment of cancer.
79. Treg cell depletion therapy for use according to any one of clauses 77 or 78 wherein the Fas axis antagonist is an anti-FasL antibody.
80. The combination of a) a priming dose of a Treg cell depletion therapy; b) a booster dose of Treg cell depletion therapy and c) a dose of a Fas axis antagonist for use in treating or preventing relapse of cancer in a subject, wherein the priming dose of the Treg cell depletion therapy and a first booster dose of the Treg cell depletion therapy are administered before a first dose of the Fas axis antagonist.
81. The combination for use according to clauses 80 further comprising d) a priming dose of a cancer vaccine, wherein the priming dose of the cancer vaccine is administered between the Treg cell depletion therapy and a first booster dose of the Treg cell depletion therapy.
82. The combination for use according to anyone of clauses 80 or 81 wherein the treatment comprises repeated administration of the booster dose of the Treg cell depletion therapy and the Fas axis antagonist, wherein each booster dose of the Treg cell depletion therapy is administered before each dose of the Fas axis antagonist.
83. The combination for use according to clause 82 further comprising e) a booster dose of a cancer vaccine, wherein each booster dose of the cancer vaccine is administered between each booster dose of the Treg cell depletion therapy and each dose of the Fas axis antagonist.
84. The combination for use according to any one of clauses 80 to 83 wherein the Treg cell depletion therapy comprises an anti-CD25 antibody and the Fas axis antagonist is an anti-FasL antibody.
85. The combination for use according to any one of clauses 81 or 83 wherein the cancer vaccine is a cytokine-expressing cellular immunotherapy, preferably wherein the cytokine-expressing cellular immunotherapy comprises GM-CSF-expressing tumor cells.
86. The T cell depletion therapy for use according to any one of clauses 33 or 77 to 79, the anti-CD25 antibody for use according to clause 34, the Fas axis antagonist for use according to any one of clauses 35, 75-76, the anti-FasL antibody for use according to clause 36, the cancer vaccine for use according to any one of clauses 37 or 38 or the combination for use according to any one of clauses 39-56 or 80-85, wherein the cancer is a solid tumor.
87. A kit comprising:
   a) a first composition comprising a Treg cell depletion therapy; and
   b) a second composition comprising a Fas axis antagonist,
   and optionally instructions for using the compositions in a combination therapy for the treatment of cancer.
88. A kit according to clause 87 further comprising c) a third composition comprising a cancer vaccine.

It is also understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

The invention will now be further described by way of the following Examples, which are meant to serve to assist one of ordinary skill in the art in carrying out the invention and are not intended in any way to limit the scope of the invention, with reference to the Figures.

### Examples

### Mice

C57BL/6 mice (6-8-weeks old female) were obtained from Charles River Laboratories. All animal studies were performed under University College London and UK Home Office ethical approval and regulations.

### Cell Lines and Tissue Culture

The highly tumourigenic and poorly immunogenic cell line B16/BL6 mouse melanoma cells were obtained from Dr. I. J. Fidler at M. D. Anderson Cancer Center (Houston, TX). B16/BL6-expressing GM-CSF (GVAX) was used for therapy. Both cell lines have been previously described (Van Elsas et al., 1999, J Exp Med vol. 190 (3) p 355-366). B16/BL6 and GVAX cells were cultured in Roswell Park Memorial Institute (RPMI) media supplemented with 10% fetal calf serum (FCS, Sigma), 100 U/mL penicillin, 100 μg/mL streptomycin and 2 mM L-glutamine (all from Gibco), at 37° C. in a humidified atmosphere of 5% CO2.

### Therapeutic Antibodies

The production of the anti-CD25^{NIB} antibody was outsourced to Evitria AG (Switzerland). Anti-CD25^{NIB} antibody is a mouse non-IL-2 blocking anti-CD25 antibody (comprising the variable regions of clone 7D4 in a mouse IgG2a backbone as described in Solomon I et al (2020) Nature Cancer, vol 1, p 1153-1166, WO2018/167104).

Isotype control antibody (InVivoMAb polyclonal Armenian hamster IgG) (Cat. #BE0091) and anti-FasL antibody (MFL3) (Cat. #BE0319) were purchased from BioXcell.

### In Vivo Tumour Experiments

C57BL/6 mice were injected with 50,000 B16/BL6 melanoma cells to the right flank subcutaneously (s.c.) at day 0.200 μg anti-CD25^{NIB} antibody was administered intraperitoneally (i.p.) on day 5. 1×10⁶ irradiated (150 Gy) GVAX cells were injected intradermally (i.d.) on the contralateral flank on day 6, 9 and 12 post tumour inoculation.

Tumour sizes were closely measured 3-4 times a week and volumes were calculated as the product of three orthogonal diameters. Tumour growth curves were plotted using tumour volumes (mm³) against days post inoculation.

Tumour growth was closely monitored after the initial combinatorial therapy. Only tumours that responded to the initial combinatorial treatment were included for further treatments.

Isotype control antibody (200 μg i.p.) and anti-FasL (MFL3) mAb (200 μg i.p.) was given on day 28, when most tumours were shrinking or remaining at stable sizes. The second dose (also 200 μg i.p.) was given one week later. In the combination treatment group, anti-CD25^{NIB} (200 μg i.p.) was given on day 25 and day 35 to maintain Treg depletion.

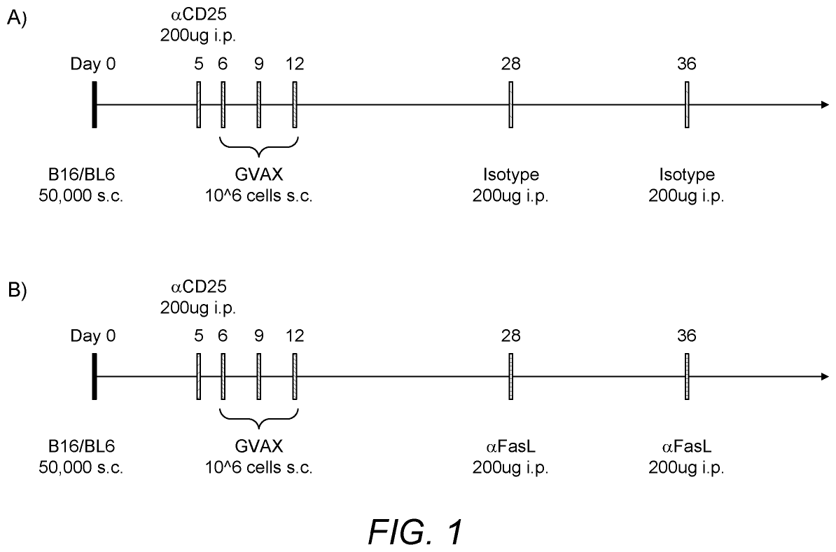

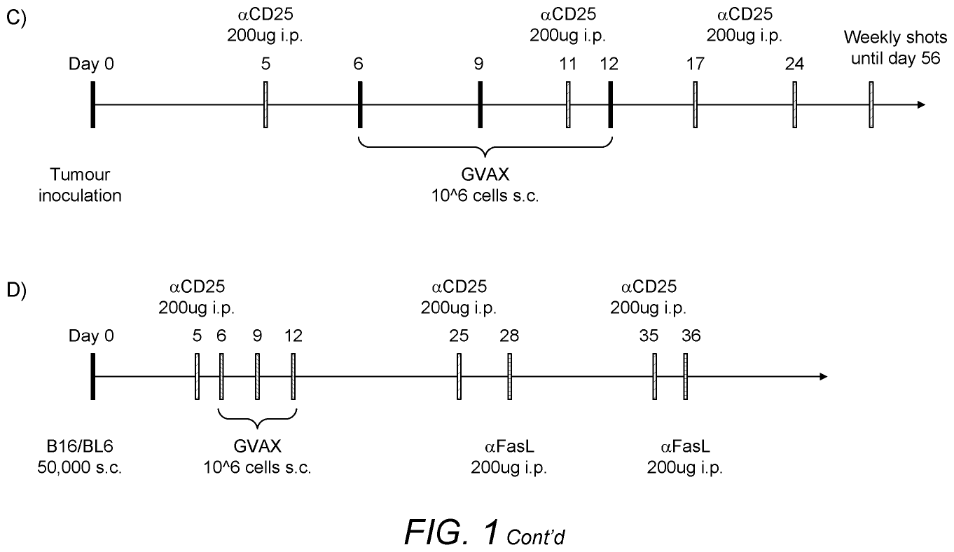

In summary the treatment groups were:
Group 1:200 μg anti-CD25^{NIB} at day 5, 1×10⁶ GVAX cells at days 6, 9 and 12, 200 μg Isotype control at days 28 and 36 (FIG. 1A).
Group 2:200 μg anti-CD25^{NIB} at day 5, 1×10⁶ GVAX cells at days 6, 9 and 12, 200 μg anti-FasL antibody at days 28 and 36 (FIG. 1B).
Group 3:200 μg anti-CD25^{NIB} at day 5, 1×10⁶ GVAX cells at days 6, 9 and 12, 200 μg anti-CD25^{NIB} at days 11, 17, 24, and weekly shots until day 56. (FIG. 1C) Group 4:200 μg anti-CD25^{NIB} at day 5, 1×10⁶ GVAX cells at days 6, 9 and 12, 200 μg anti-CD25^{NIB} at days 25 and 36; 200 μg anti-FasL antibody at days 28 and 36 (FIG. 1D).
Tumour sizes were measured 2-3 times a week. Mice were euthanized when any diameter reached 150 mm.

### Results

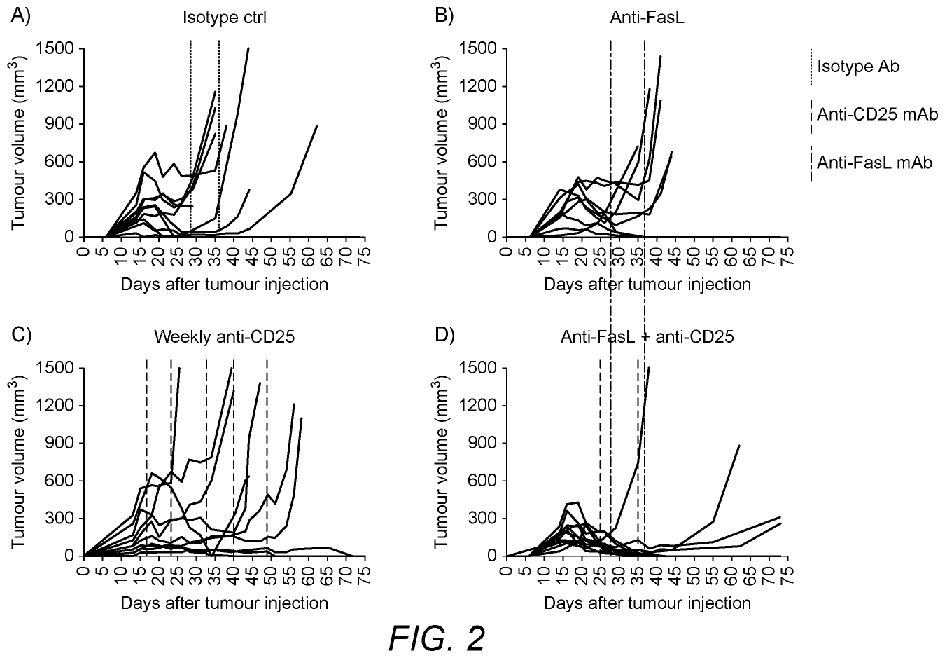

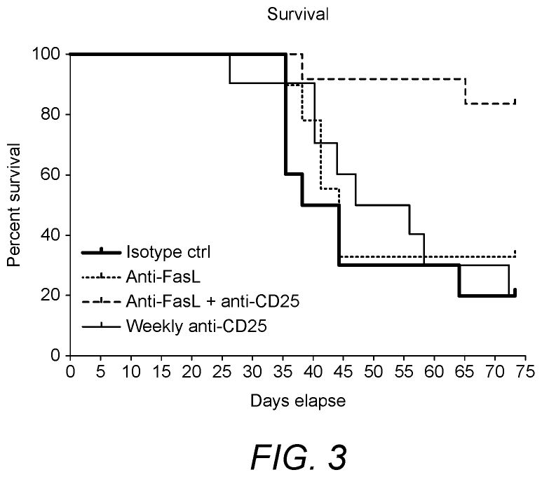

The results showing tumor growth and survival over the elapsed time are shown in FIGS. 2 and 3. These results show that the use of an anti-FasL antibody in combinational therapy with an anti-CD25 antibody significantly prevented relapse of tumor growth and improved survival.

In particular, these experiments show that the addition of a combination of anti-FasL and anti-CD25 antibody to an initial combinational therapy of anti-CD25 antibody and GVAX, prevented relapse of tumor growth and improved survival compared to the isotype control, anti-FasL antibody treatment alone and anti-CD25 antibody treatment alone.

As there is a loss of activated Teffs and a gain of Tregs over time, treating stable tumours with the enhanced therapy regimen (multiple cycles of the anti-FasL antibody and anti-CD25 antibody combination treatment) is thought to potentially provide more antigen-specific T cells and maintain low count of Tregs in the TME, thereby preventing relapse. The blockade of the Fas/FasL axis by the anti-FasL antibody is thought to inhibit Fas-mediated cell death of activated Teffs. This inhibition of Fas-mediated cell death of activated Teffs in combination with Treg depletion, helps maintain an immunogenic TME, therefore achieving a better therapeutic effect.

All publications mentioned in the above specification are herein incorporated by reference. Various modifications and variations of the described uses and methods of the invention will be apparent to those skilled in the art without departing from the scope and spirit of the invention. Although the invention has been described in connection to specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

A summary of sequences referred to in the application is provided in the table below:

TABLE 2

| SEQ | | |
|---|---|---|
| ID NO | Description of Antibody Sequences | Also referred to as: |
| 1 | MDSYLLMWGL LTFIMVPGCQ AELCDDDPPE IPHATFKAMA | Human CD25 sequence |
| | YKEGTMLNCE CKRGFRRIKS GSLYMLCTGN SSHSSWDNQC | |
| | QCTSSATRNT TKQVTPQPEE QKERKTTEMQ SPMQPVDQAS | |
| | LPGHCREPPP WENEATERIY HFVVGQMVYY QCVQGYRALH | |
| | RGPAESVCKM THGKTRWTQP QLICTGEMET SQFPGEEKPQ | |
| | ASPEGRPESE TSCLVTTTDF QIQTEMAATM ETSIFTTEYQ | |
| | VAVAGCVFLL ISVLLLSGLT WQRRQRKSRR TI | |
| 2 | GTFSSLAIS | CDR-H1 aCD25-a-686 |
| | | CDR-H1 aCD25-a-686-m1 |
| | | CDR-H1 aCD25-a-686-m3 |
| 3 | GTFSSLAIT | CDR-H1 aCD25-a-686-m2 |
| 4 | GTFSALAIS | CDR-H1 aCD25-a-686-m4 |
| 5 | GTFSSLAIF | CDR-H1 aCD25-a-686-m5 |
| 6 | GIIPIFGTANYAQKFQG | CDR-H2 aCD25-a-686 |
| 7 | AIIPVFGTASYAQKFQG | CDR-H2 aCD25-a-686-m1 |
| 8 | GIIPIFGDASYAQKFQG | CDR-H2 aCD25-a-686-m2 |
| 9 | GIIPIFGDANYAQKLQG | CDR-H2 aCD25-a-686-m3 |
| 10 | GIIPLFGRANYAQKFQG | CDR-H2 aCD25-a-686-m4 |
| 11 | GIIPVFGQANYAQKFQG | CDR-H2 aCD25-a-686-m5 |
| 12 | ARGGSVSGTLVDFDI | CDR-H3 aCD25-a-686 |
| | | CDR-H3 aCD25-a-686-m1 |
| | | CDR-H3 aCD25-a-686-m2 |
| | | CDR-H3 aCD25-a-686-m3 |
| | | CDR-H3 aCD25-a-686-m4 |
| | | CDR-H3 aCD25-a-686-m5 |
| 13 | RASQSISSWLA | CDR-L1 aCD25-a-686 |
| | | CDR-L1 aCD25-a-686-m1 |
| | | CDR-L1 aCD25-a-686-m2 |
| | | CDR-L1 aCD25-a-686-m3 |
| | | CDR-L1 aCD25-a-686-m4 |
| | | CDR-L1 aCD25-a-686-m5 |
| 14 | KASSLES | CDR-L2 aCD25-a-686 |
| | | CDR-L2 aCD25-a-686-m1 |
| | | CDR-L2 aCD25-a-686-m2 |
| | | CDR-L2 aCD25-a-686-m3 |
| | | CDR-L2 aCD25-a-686-m4 |
| | | CDR-L2 aCD25-a-686-m5 |
| 15 | QQYNIYPIT | CDR-L3 aCD25-a-686 |
| | | CDR-L3 aCD25-a-686-m1 |
| | | CDR-L3 aCD25-a-686-m2 |
| | | CDR-L3 aCD25-a-686-m3 |
| | | CDR-L3 aCD25-a-686-m4 |
| | | CDR-L3 aCD25-a-686-m5 |
| 16 | QVQLVQSGAEVKKPGSSVKVSCKASGGTFSSLAISWVRQAPGQGL | VH aCD25-a-686 |
| | EWMGGIIPIFGTANYAQKFQGRVTITADESTSTAYMELSSLRSED | |
| | TAVYYCARGGSVSGTLVDFDIWGQGTMVTVSS | |
| 17 | QVQLVQSGAEVKKPGSSVKVSCKASGGTFSSLAISWVRQAPGQGL | VH aCD25-a-686-m1 |
| | EWMGAIIPVFGTASYAQKFQGRVTITADESTSTAYMELSSLRSED | |
| | TAVYYCARGGSVSGTLVDFDIWGQGTMVTVSS | |
| 18 | QVQLVQSGAEVKKPGSSVKVSCKASGGTFSSLAITWVRQAPGQGL | VH aCD25-a-686-m2 |
| | EWMGGIIPIFGDASYAQKFQGRVTITADESTSTAYMELSSLRSED | |
| | TAVYYCARGGSVSGTLVDFDIWGQGTMVTVSS | |
| 19 | QVQLVQSGAEVKKPGSSVKVSCKASGGTFSSLAISWVRQAPGQGL | VH aCD25-a-686-m3 |
| | EWMGGIIPIFGDANYAQKLQGRVTMTTDTSTSTAYMELRSLRSDD | |
| | TAVYYCARGGSVSGTLVDFDIWGQGTMVTVSS | |
| 20 | QVQLVQSGAEVKKPGSSVKVSCKASGGTFSALAISWVRQAPGQGL | VH aCD25-a-686-m4 |
| | EWMGGIIPLFGRANYAQKFQGRVTITADESTSTAYMELSSLRSED | |
| | TAVYYCARGGSVSGTLVDFDIWGQGTMVTVSS | |
| 21 | QVQLVQSGAEVKKPGSSVKVSCKASGGTFSSLAIFWVRQAPGQGL | VH aCD25-a-686-m5 |
| | EWMGGIIPVFGQANYAQKFQGRVTITVDESTSTAYMELSSLRSED | |
| | TAVYYCARGGSVSGTLVDFDIWGQGTMVTVSS | |
| 22 | DIQMTQSPSTLSASVGDRVTITCRASQSISSWLAWYQQKPGKAPK | VL aCD25-a-686 |
| | LLIYKASSLESGVPSRFSGSGSGTEFTLTISSLQPDDFATYYCQQ | VL aCD25-a-686-m1 |
| | YNIYPITFGGGTKVEIK | VL aCD25-a-686-m2 |
| | | VL aCD25-a-686-m3 |
| | | VL aCD25-a-686-m4 |
| | | LC aCD25-a-686-m5 |
| 23 | YTFTSYYMH | CDR-H1 aCD25-a-674 |
| 24 | IINPSGGSTSYAQKFQG | CDR-H2 aCD25-a-674 |
| 25 | ARGGAEYIPAEYFQH | CDR-H3 aCD25-a-674 |
| 26 | RASQSVSSYLA | CDR-L1 aCD25-a-674 |
| 27 | DASNRAT | CDR-L2 aCD25-a-674 |
| 28 | QQRPFLPT | CDR-L3 aCD25-a-674 |
| 29 | QVQLVQSGAEVKKPGASVKVSCKASGYTFTSYYMHWVRQAPGQGL | VH aCD25-a-674 |
| | EWMGIINPSGGSTSYAQKFQGRVTMTRDTSTSTVYMELSSLRSED | |
| | TAVYYCARGGAEYIPAEYFQHWGQGTLVTVSS | |
| 30 | EIVMTQSPATLSLSPGERATLSCRASQSVSSYLAWYQQKPGQAPR | VL aCD25-a-674 |
| | LLIYDASNRATGIPARFSGSGSGTDFTLTISSLEPEDFAVYYCQQ | |
| | RPFLPTFGGGTKVEIK | |
| 31 | FTFSSYGMH | CDR-H1 aCD25-a-646 |
| 32 | FTFASYGMH | CDR-H1 aCD25-a-646-m1 |
| 33 | FTFPSYGMH | CDR-H1 aCD25-a-646-m2 |
| | | CDR-H1 aCD25-a-646-m3 |
| | | CDR-H1 aCD25-a-646-m4 |
| | | CDR-H1 aCD25-a-646-m5 |
| 34 | VIWYDGSNKGYADSVKG | CDR-H2 aCD25-a-646 |
| 35 | VIWYDASTKYYADSVKG | CDR-H2 aCD25-a-646-m1 |
| | | CDR-H2 aCD25-a-646-m4 |
| 36 | VIWYDAINKYYADSVKG | CDR-H2 aCD25-a-646-m2 |
| 37 | VIWYDAVNKYYADSVKG | CDR-H2 aCD25-a-646-m3 |
| 38 | VIWYDALNKYYADSVKG | CDR-H2 aCD25-a-646-m5 |
| 39 | ARDLGYGDYAAHDY | CDR-H3 aCD25-a-646 |
| | | CDR-H3 aCD25-a-646-m1 |
| | | CDR-H3 aCD25-a-646-m2 |
| | | CDR-H3 aCD25-a-646-m3 |
| | | CDR-H3 aCD25-a-646-m4 |
| | | CDR-H3 aCD25-a-646-m5 |
| 40 | RASQSISSWLA | CDR-L1 aCD25-a-646 |
| | | CDR-L1 aCD25-a-646-m1 |
| | | CDR-L1 aCD25-a-646-m2 |
| | | CDR-L1 aCD25-a-646-m3 |
| | | CDR-L1 aCD25-a-646-m4 |
| | | CDR-L1 aCD25-a-646-m5 |
| 41 | KASSLES | CDR-L2 aCD25-a-646 |
| | | CDR-L2 aCD25-a-646-m1 |
| | | CDR-L2 aCD25-a-646-m2 |
| | | CDR-L2 aCD25-a-646-m3 |
| | | CDR-L2 aCD25-a-646-m4 |
| | | CDR-L2 aCD25-a-646-m5 |
| 42 | QQHNTHPYT | CDR-L3 aCD25-a-646 |
| | | CDR-L3 aCD25-a-646-m1 |
| | | CDR-L3 aCD25-a-646-m2 |
| | | CDR-L3 aCD25-a-646-m3 |
| | | CDR-L3 aCD25-a-646-m4 |
| | | CDR-L3 aCD25-a-646-m5 |
| 43 | QVQLVESGGGVVQPGRSLRLSCAASGFTFSSYGMHWVRQAPGKGL | VH aCD25-a-646 |
| | EWVAVIWYDGSNKGYADSVKGRFTISRDNSKNTLYLQMNSLRAED | |
| | TAVYYCARDLGYGDYAAHDYWGQGTLVTVSS | |
| 44 | EVQLVESGGGLVKPGGSLRLSCAASGFTFASYGMHWVRQAPGKGL | VH aCD25-a-646-m1 |
| | EWVAVIWYDASTKYYADSVKGRFTISRDNSKNTLYLQMNSLRAED | |
| | TAVYYCARDLGYGDYAAHDYWGQGTLVTVSS | |
| 45 | QVQLVESGGGLVQPGGSLRLSCAASGFTFPSYGMHWVRQAPGKGL | VH aCD25-a-646-m2 |
| | EWVAVIWYDAINKYYADSVKGRFTISRDNSKNTLYLQMNSLRAED | |
| | TAVYYCARDLGYGDYAAHDYWGQGTLVTVSS | |
| 46 | QVQLVESGGGVVQPGRSLRLSCAASGFTFPSYGMHWVRQAPGKGL | VH aCD25-a-646-m3 |
| | EWVAVIWYDAVNKYYADSVKGRFTISRDNSKNTLYLQMNSLRAED | |
| | TAVYYCARDLGYGDYAAHDYWGQGTLVTVSS | |
| 47 | QVQLVESGGGVVQPGRSLRLSCAASGFTFPSYGMHWVRQAPGKGL | VH aCD25-a-646-m4 |
| | EWVAVIWYDASTKYYADSVKGRFTISRDNSKNTLYLQMNSLRAED | |
| | TAVYYCARDLGYGDYAAHDYWGQGTLVTVSS | |
| 48 | EVQLLESGGGLVQPGGSLRLSCAASGFTFPSYGMHWVRQAPGKGL | VH aCD25-a-646-m5 |
| | EWVAVIWYDALNKYYADSVKGRFTISRDNSKNTLYLQMNSLRAED | |
| | TAVYYCARDLGYGDYAAHDYWGQGTLVTVSS | |
| 49 | DIQMTQSPSTLSASVGDRVTITCRASQSISSWLAWYQQKPGKAPK | VL aCD25-a-646 |
| | LLIYKASSLESGVPSRFSGSGSGTEFTLTISSLQPDDFATYYCQQ | VL aCD25-a-646-m1 |
| | HNTHPYTFGGGTKVEIK | VL aCD25-a-646-m2 |
| | | VL aCD25-a-646-m3 |
| | | VL aCD25-a-646-m4 |
| | | VL aCD25-a-646-m5 |
| 50 | QVQLVQSGAE VKKPGSSVKV SCKASGGTFS SLAISWVRQA | HC sequence of RG6292 |
| | PGQGLEWMGG IIPIFGTANY AQKFQGRVTI TADESTSTAY | |
| | MELSSLRSED TAVYYCARGG SVSGTLVDFD IWGQGTMVTV | |
| | SSASTKGPSV FPLAPSSKST SGGTAALGCL VKDYFPEPVT | |
| | VSWNSGALTS GVHTFPAVLQ SSGLYSLSSV VTVPSSSLGT | |
| | QTYICNVNHK PSNTKVDKKV EPKSCDKTHT CPPCPAPELL | |
| | GGPSVFLFPP KPKDTLMISR TPEVTCVVVD VSHEDPEVKF | |
| | NWYVDGVEVH NAKTKPREEQ YNSTYRVVSV LTVLHQDWLN | |
| | GKEYKCKVSN KALPAPIEKT ISKAKGQPRE PQVYTLPPSR | |
| | DELTKNQVSL TCLVKGFYPS DIAVEWESNG QPENNYKTTP | |
| | PVLDSDGSFF LYSKLTVDKS RWQQGNVFSC SVMHEALHNH | |
| | YTQKSLSLSP GK | |
| 51 | DIQMTQSPST LSASVGDRVT ITCRASQSIS SWLAWYQQKP | LC sequence of RG6292 |
| | GKAPKLLIYK ASSLESGVPS RFSGSGSGTE FTLTISSLQP | |
| | DDFATYYCQQ YNIYPITFGG GTKVEIKRTV AAPSVFIFPP | |
| | SDEQLKSGTA SVVCLLNNFY PREAKVQWKV DNALQSGNSQ | |
| | ESVTEQDSKD STYSLSSTLT LSKADYEKHK VYACEVTHQG | |
| | LSSPVTKSFN RGEC | |
| 52 | YQCVQGYRALHRGP | amino acids 150-163 of |
| | | SEQ ID NO: 1 |
| 53 | SVCKMTHGKTRWTQPQLICTG | amino acids 166-186 of |
| | | SEQ ID NO: 1 |
| 54 | KEGTMLNCECKRGER | amino acids 42-56 of SEQ |
| | | ID NO: 1 |
| 55 | NSSHSSWDNQCQCTSSATR | amino acids 70-88 of SEQ |
| | | ID NO: 1 |
| 56 | YQCVQGYRA | amino acids 150-158 of |
| | | SEQ ID NO: 1 |
| 57 | RWTQP | amino acids 176-180 of |
| | | SEQ ID NO: 1 |
| 58 | SSWDNQCQCTS | amino acids 74-84 of SEQ |
| | | ID NO: 1 |
| 59 | NSSHSSWDNQCQCTS | amino acids 70-84 of SEQ |
| | | ID NO: 1 |

1. A combination of a Treg cell depletion therapy and a Fas axis antagonist for use in the treatment of cancer, wherein the Treg cell depletion therapy and the Fas axis antagonist are for separate, simultaneous or sequential administration.

2. The combination for use according to claim 1 wherein the Treg cell depletion therapy comprises an anti-CD25 antibody.

3. The combination for use according to claim 1 or 2 further comprising a cancer vaccine.

4. The combination for use according to claim 3 wherein the combination comprises a) a first component which comprises the anti-CD25 antibody, b) a second component which comprises the Fas axis antagonist and c) a third component which comprises the cancer vaccine for separate, simultaneous or sequential administration.

5. The combination for use according to claim 4 wherein the Fas axis antagonist is for administration after the anti-CD25 antibody and the cancer vaccine.

6. The combination for use according any one of claims 4 to 5 wherein the Fas axis antagonist is for administration after depletion of Treg cells in the subject by administration of the anti-CD25 antibody.

7. The combination for use according any one of claims 4 to 6 wherein the administration of the anti-CD25 antibody to the subject, is followed by administration of the cancer vaccine, and then followed by administration of the Fas axis antagonist.

8. The combination for use according to any one of claims 4 to 7 wherein the treatment comprises administration of multiple doses of the anti-CD25 antibody.

9. The combination for use according to any one of claims 4 to 8 wherein the treatment comprises administration of multiple doses of the Fas axis antagonist.

10. The combination for use according to any one of claims 4 to 9 wherein the treatment comprises administration of multiple doses of the cancer vaccine.

11. The combination for use according to any one of claims 8 to 10 wherein the administration comprises:
a first administration of the anti-CD25 antibody;
followed by administration of one or more doses of the cancer vaccine;
followed by administration of one or more alternating doses of the anti-CD25 antibody and Fas axis antagonist, wherein each further dose of the anti-CD25 antibody is for administration before the dose of the Fas axis antagonist.

12. The combination for use according to claim 11 wherein the administration of the combination of the dose of a Fas axis antagonist and the further dose of the anti-CD25 antibody is repeated in a treatment cycle of at least 7 days.

13. The combination for use according to any one of claim 11 or 12 wherein the administration of the one or more alternating doses of the anti-CD25 antibody and Fas axis antagonist also comprises administration of one or more doses of the cancer vaccine, wherein each further dose of the anti-CD25 antibody and the cancer vaccine is for administration before the dose of the Fas axis antagonist.

14. The combination for use according to any one of claims 4 to 13 wherein the anti-CD25 antibody inhibits less than 50% of the signalling of IL-2 via CD25 compared to IL-2 signalling in the absence of the antibody.

15. The combination for use according to any one of claims 4 to 14 wherein the cancer vaccine is a cytokine-expressing cellular immunotherapy.

16. The combination for use according claim 15 wherein the cytokine-expressing cellular immunotherapy comprises GM-CSF-expressing tumor cells.

17. The combination for use according to any one of claims 1 to 16 wherein the Fas axis antagonist is an anti-FasL antibody.

18. The combination for use according to any one of claims 1 to 17 wherein the cancer is a solid tumour.

19. A kit for the treatment of cancer comprising:
a) a first composition comprising a Treg cell depletion therapy;
b) a second composition comprising a Fas axis antagonist and optionally instructions for use in a combination therapy.

20. A kit according to claim 19 wherein the kit further comprises c) a third composition comprising a cancer vaccine.
